(19) United States
(12) Patent Application Publication
(10) Pub. No.: US 2026/0151606 A1
(43) Pub. Date: Jun. 4, 2026

(54) Microneedle Patch, Microneedle Mold and Preparation Method for Microneedle Patch

(71) Applicants: YOUWE (ZHUHAI) BIOTECHNOLOGY CO., LTD, Zhuhai, Guangdong (CN); CREWAY (ZHUHAI) PHARMACEUTICAL TECHNOLOGY CO., LTD, Zhuhai, Guangdong (CN)

(72) Inventors: Gang LENG, Zhuhai, Guangdong (CN); Chengguo LI, Zhuhai, Guangdong (CN); Yonghao MA, Zhuhai, Guangdong (CN); Jinyong CHEN, Zhuhai, Guangdong (CN); Lianhua CHEN, Zhuhai, Guangdong (CN); Yanfen LI, Zhuhai, Guangdong (CN)

(21) Appl. No.: 18/717,218

(22) PCT Filed: Dec. 8, 2022
(86) PCT No.: PCT/CN2022/137461

§ 371 Date: Jun. 6, 2024

(30) Foreign Application Priority Data
Dec. 14, 2021 (CN) 202111530647.1
Dec. 14, 2021 (CN) 202111532216.9
Dec. 14, 2021 (CN) 202111532220.5

(51) Int. Cl. B29C 33/38; A61M 37/00; B29C 45/26; B29C 45/56

(52) U.S. Cl. CPC A61M 37/0015; A61M 2037/0023; A61M 2037/0046; A61M 2037/0053; A61M 2202/0007; A61M 2202/06; A61M 2207/10; A61M 2210/04

(57) ABSTRACT

A microneedle patch, including: a substrate layer and a first microneedle provided on the substrate layer; wherein the number of the first microneedle is at least one, the first microneedle includes a microneedle body and at least one protrusion provided on the microneedle body; and the each protrusion extends in a radial direction of the first microneedle from a needle tip to the substrate layer, and has a tip end away from the microneedle body.

### CROSS-REFERENCE TO RELATED APPLICATION(S)

The present disclosure is a national stage application of International Patent Application No. PCT/CN2022/137461, which is filed on Dec. 8, 2022 and is incorporated by reference in its entirety. The International Patent Application claims priority to Chinese Application No. 202111532216.9, filed in the Chinese Patent Office on Dec. 14, 2021, and entitled “Microneedle Patch With Easy-to-tear Substrate”, Chinese Application No. 202111530647.1, filed in the Chinese Patent Office on Dec. 14, 2021, and entitled “Microneedle Patch”, and Chinese Application No. 202111532220.5, filed in the Chinese Patent Office on Dec. 14, 2021, and entitled “Microneedle Mold and Preparation Method for Microneedle Patch”, all of which are incorporated by reference in their entirety.

### TECHNICAL FIELD

The present disclosure belongs to the technical field of microneedles, and in particular, to a microneedle patch, a microneedle mold and a preparation method for the microneedle patch.

### BACKGROUND

In recent hundred years, metal injection needles, as mainstream appliances for medicine injection, have been widely used in the medical industry, but the pain and fear brought about by the use of metal injection needles make it difficult for some people (especially children) to accept. Along with a progress and development of technology, microneedle patches for medicine injection emerged.

The microneedle patch generally includes a substrate layer and microneedles arranged on the substrate layer, and the microneedles can be dissolved. During use, a side of the microneedle patch provided with the microneedles is pressed against the skin, until the microneedles puncture into the skin and the substrate layer is attached to the skin. Since the length of the microneedles is short, the microneedles will not cause nerve injury and pain to the patient during puncturing into the skin, and thus are gradually recognized by the public.

In order to stably embed the microneedles into the skin, circumferential surfaces of the existing microneedles are non-smooth surfaces, to improve gripping force between the microneedles and the skin, so that the microneedles are stably embedded into the skin until the microneedles are dissolved, thereby improving the administration effect of the microneedles. After the microneedles puncture into the skin, since the skin has both elasticity and rigidity, the microneedles cannot be well attached to the skin, and the skin is prone to apply a force to the non-smooth surfaces of the microneedles, and the microneedles are squeezed out, resulting in a poor anchoring effect of the microneedles in the skin.

### SUMMARY

In order to solve the described technical problems, the present disclosure provides a microneedle patch, a microneedle mold and a preparation method for the microneedle patch, which solve problems in the related art that microneedles are not stably embedded in the skin after the microneedles puncture into the skin and the microneedles are easily squeezed out of the skin.

The present disclosure adopts the following technical solutions:
a microneedle patch, including: a substrate layer and a first microneedle provided on the substrate layer, wherein the number of the first microneedle is at least one; the first microneedle includes a microneedle body and at least one fixing portion provided on the microneedle body; the fixing portion includes protrusions, the protrusions extend in a radial direction of the first microneedle from a needle tip to a needle base, and each have a tip end away from the microneedle body.

The first microneedle contains a medicine component, and the first microneedle can be dissolved in skin.

After the first microneedle punctures into the skin, the substrate layer is torn off; the number of the protrusions is greater than or equal to two, and along the direction in which the needle base of the first microneedle extends towards the needle tip, the horizontal projection area and volume of the plurality of protrusions increase in sequence. Specifically, an included angle between the extension direction of each protrusion and an axis of the microneedle body is α, 15°≤α≤75, and more preferably, 30°≤α≤60°.

Specifically, when the number of the protrusions is three, along the direction in which the needle base of the microneedle extends towards the needle tip, the protrusions sequentially include a first protrusion, a second protrusion and a third protrusion; and an included angle between the extension direction of the first protrusion and an axis of the microneedle body is 45°, an included angle between the extension direction of the second protrusion and the axis of the microneedle body is 30°, and an included angle between the extension direction of the third protrusion and the axis of the microneedle body is 60°.

Specifically, when the number of the protrusions is two, the protrusions sequentially include a first protrusion and a second protrusion; a circumferential side wall of the first protrusion extends vertically, or is obliquely arranged in a direction close to the microneedle body; a circumferential side wall of the second protrusion extends vertically, or is obliquely arranged in a direction away from the microneedle body, and abuts against a circumferential side wall of a mold molding cavity; and when the number of the protrusions is three, the protrusions sequentially include a first protrusion, a second protrusion and a third protrusion; a circumferential side wall of the first protrusion extends vertically; a circumferential side wall of the second protrusion extends vertically, or is obliquely arranged in a direction close to the microneedle body; and a circumferential side wall of the third protrusion extends vertically, or is obliquely arranged in a direction away from the microneedle body, and abuts against a circumferential side wall of the mold molding cavity.

The horizontal projection of the needle base of the first microneedle is fan-shaped and the microneedle body is a cone with a horizontal projection in a fan shape, the protrusions are located on an edge of the microneedle body connected to the tip end of a fan-shaped flat surface, and the protrusions on the microneedle body face the same direction; or the microneedle body is a cone with a horizontal projection in an elliptic fan shape, the protrusions are provided on an edge of the microneedle body connected to the tip end of an elliptic fan-shaped bottom face and are located on a short-axis side of the horizontal projection of the microneedle body; or the microneedle body is a pyramid, and the protrusions are located on an edge of the microneedle body.

Specifically, the microneedle patch further includes a second microneedle; at least one first microneedle and at least one second microneedle arranged at an interval form a microneedle unit; the microneedle patch includes at least one microneedle unit, the second microneedle is a microneedle not provided with the fixing portion, and the second microneedle is located on the side of the first microneedle not provided with the fixing portion.

A stress point structure for tearing the substrate layer and separating the first microneedle from the substrate layer is provided at a connection position between the first microneedle and the substrate layer; the stress point structure includes a substrate through-hole provided on the substrate layer and on the same side as the fixing portion on the first microneedle.

The stress point structure further includes guide ports; the guide ports are located at junctions of the needle base of the first microneedle and the substrate through-hole, and are located on a circumferential side wall of the needle base or on the substrate layer.

A minimum length of a connecting line of the guide ports on two sides of the first microneedle is greater than a maximum length of the second microneedle in a direction parallel to the connecting line of the guide ports.

After the first microneedle punctures into the skin and when the substrate layer is torn off, the first microneedle and the substrate through-hole adjacent thereto form a group of stress units, and at least one group of stress units are arranged at intervals on the substrate layer; the substrate layer and the first microneedle are separated by the stress point structure to form cracks, the cracks intersect on the substrate layer between two adjacent groups of stress units; or after the microneedle unit punctures into the skin and when the substrate layer is torn off, the microneedle unit and the substrate through-hole adjacent thereto form a group of stress units, and at least one group of stress units are arranged at intervals on the substrate layer; after the microneedle punctures into the skin and when the substrate layer is torn off, the cracks formed by the substrate layer being separated from the microneedle unit by the stress point structure intersect at a gap between two adjacent groups of microneedle units.

The stress point structure further includes guide ports; the cracks formed by separating the substrate layer from the microneedle unit extend along the guide ports from the first microneedle to the second microneedle, and extends along a circumferential side wall of the needle base of the second microneedle, so that the second microneedle is separated from the substrate layer.

Extension lines of the guide ports intersect with or are tangent to a circumferential side wall of the needle base of the second microneedle. A microneedle mold, applied to manufacturing a microneedle patch, sequentially comprising an upper mold and a lower mold from top to bottom, the upper mold is detachably connected to the lower mold, the upper mold includes an upper mold bottom plate, and at least one inner mold is provided at the bottom of the upper mold bottom plate; at least one first molding cavity is provided at the top of the lower mold, each first molding cavity is provided corresponding to at least one inner mold, and the first molding cavity accommodates the inner mold; the inner mold includes an inner mold body and at least one groove provided on the inner mold body, and the groove is used for forming a protrusion on a circumferential surface of a first microneedle; and a space enclosed by the first molding cavity and the inner mold is used for molding the first microneedle.

The inner mold is provided with guide port molds to form guide ports on the microneedle provided with the protrusions after the first microneedle is molded.

The guide port molds are protuberances provided on the inner mold body close to a maximum diameter end face and extending in a circumferential direction, so as to form the guide ports on the first microneedle provided with the protrusions.

The length of a connecting line between two ends of open ends of a maximum diameter end face of the guide port molds provided on the inner mold body is greater than the length of a connecting line of two joint points between the two guide ports on the microneedle and an outer circumferential side wall of the needle base on the microneedle provided with the protrusions.

A direction from an end with the smallest diameter to an end with the largest diameter of the inner mold body is a Y direction, and the grooves extend along the Y direction.

The number of the grooves is at least two, and along a direction opposite to the Y direction, the volumes of the grooves increase sequentially, so that the volumes of corresponding protrusions in the molded microneedle increase sequentially.

The microneedle mold further includes a middle plate, wherein the middle plate is located between the upper mold and the lower mold, the middle plate is provided with a middle plate through-hole extending vertically, the middle plate through-hole accommodates the inner mold, the middle plate through-hole is used to form a substrate layer, and the substrate layer and the microneedle form a microneedle patch.

At least one second molding cavity is further provided at the top of the lower mold, and when the microneedle mold is closed, no inner mold is provided in the second molding cavity; and a space in the second molding cavity is used for molding a second microneedle not provided with the protrusions.

The first molding cavity, the inner mold embedded with the first molding cavity, and the second molding cavity form a microneedle mold unit, the microneedle mold unit is used for molding a microneedle unit, and when the microneedle mold is closed, the second molding cavity is located at the side, provided with the grooves, of the inner mold embedded with the first molding cavity.

The upper mold bottom plate is provided with at least one upper mold bottom plate through-hole, each of the upper mold bottom plate through-hole is provided adjacent to a corresponding inner mold, and is provided at the same side as the grooves on the inner mold; when the inner mold is located in the first molding cavity, along the direction in which the lower mold extends towards the upper mold, the space enclosed by the first molding cavity and the inner mold extends linearly and penetrates through the upper mold bottom plate to form the upper mold bottom plate through-hole.

The first molding cavity has a radius R1 and the second molding cavity has a radius r1, and then 0.17R₁≤r₁≤0.59R₁.

Along a connecting line direction between the center of circle of the first molding cavity and the center of circle of the second molding cavity, the distance from the center of circle of the first molding cavity of a microneedle mold unit to the center of circle of the first molding cavity of an adjacent microneedle unit is L1, then 3R₁≤L₁≤6R₁.

A preparation method for a microneedle patch, which is prepared by a microneedle mold, wherein the preparation method includes the following steps:
injecting a formulation liquid into a microneedle molding cavity formed by the upper mold and the lower mold; and drying for 1-3 h at 4-30° C. under a humidity condition of 20%-60%, so as to dry and mold the microneedle; wherein the microneedle molding cavity at least includes a first molding cavity, the first molding cavity accommodates an inner mold on the upper mold, and the inner mold is used for molding protrusions on the first microneedle.

Further, injecting a formulation liquid into a microneedle molding cavity formed by the upper mold and the lower mold includes the following steps:
S1: injecting a formulation liquid into the first molding cavity; and
S2: combining the upper mold and the lower mold, so that the inner mold is inserted into the first molding cavity; or, S1: combining the upper mold and the lower mold, so that the inner mold is inserted into the first molding cavity; and S2: injecting the formulation liquid into a space enclosed by the first molding cavity and the inner mold.

The step S2 further includes: providing a middle plate between the upper mold and the lower mold, a middle plate through-hole on the middle plate accommodating the inner mold, injecting the formulation liquid into the middle plate through-hole, and the formulation liquid in the middle plate through-hole drying and molding a substrate layer; the formulation liquid in a space enclosed by the first molding cavity and the inner mold drying and molding the microneedle provided with the protrusions; pulling the dried microneedle provided with the protrusions out of the first molding cavity; and when the microneedle provided with the protrusions is pulled out of the first molding cavity, peeling off the substrate layer from the side of the microneedle not provided with the protrusions to the side of the microneedle provided with the protrusions, to separate the microneedle provided with the protrusions from the first molding cavity, so as to mold a microneedle patch.

The step S2 further includes: drying and molding the microneedle provided with the protrusions; pulling the dried microneedle provided with the protrusions out of the first molding cavity; and when the microneedle provided with the protrusions is pulled out of the first molding cavity, attaching an adhesive substrate layer to the top of the lower mold, so that the adhesive substrate layer adheres to the end part of the end of the microneedle away from the needle tip, and peeling off the adhesive substrate layer from the side of the microneedle not provided with the protrusions to the side of the microneedle provided with the protrusions, to separate the microneedle provided with the protrusions from the first molding cavity, so as to mold a microneedle patch.

Beneficial effects of some embodiments of the present disclosure are:
1. The microneedle patch of the embodiment includes a substrate layer and a microneedle provided on the substrate layer; by providing protrusions on the microneedle, the protrusions extend along a radial direction of the microneedle from the needle tip to the substrate layer, so that after the microneedle punctures into and enters the skin, the microneedle is anchored to the skin by the protrusions, which increases the gripping force between the microneedle and the skin, and prevents the microneedle from being detached from the skin due to elastic deformation and pressing of the skin, thereby achieving continuous and accurate administration, and ensuring the administration effect.

During use, after the microneedle punctures into the skin, the protrusions and/or the part of the needle tip protruding from the needle base on the microneedle are embedded into the skin, to increase the gripping force between the microneedle and the skin, such that the substrate layer can be immediately torn off after the microneedle punctures into the skin, improving the comfort and aesthetics of the microneedle patch during use, especially, for situations of scratching or licking the substrate layer in an administration process of a child, a pet and a psychopath, the microneedle is prevented from falling off. In the process of tearing off and removing the substrate layer, since the protrusions are anchored in the skin, the gripping force between the microneedle and the skin is increased, so that it can be ensured that when the substrate layer is removed, the microneedle is not detached from the skin due to moving along with the substrate layer.
2. When the microneedle patch is used, after the microneedle punctures into the skin, a hand grips the substrate layer, and peels off the substrate layer along the direction from the side of the microneedle provided with the fixing portion to the side not provided with the fixing portion. A stress point structure for tearing the substrate layer and separating the microneedle from the substrate layer is provided at a connection position between the microneedle and the substrate layer; there is large stress concentration at the stress point structure, when the substrate layer is to be torn off, a critical stress of breaking and separating the substrate layer from the microneedle at the stress point structure is small, and therefore under the action of a small external force, the substrate layer is broken and separated from the microneedle, so as to tear off the substrate layer. includes an upper mold and a lower mold from top to bottom, wherein the upper mold and the lower mold are detachably connected, the upper mold includes an upper mold bottom plate, and at least one inner mold is provided at the bottom of the upper mold bottom plate. At least one first molding cavity is provided at the top of the lower mold, each first molding cavity is provided corresponding to at least one inner mold, and the first molding cavity accommodates the inner mold. The inner mold is provided with grooves, so as to form protrusions on a circumferential surface of the microneedle. A space enclosed by the first molding cavity and the inner mold is used for molding the microneedle. After the microneedle is molded, first the upper mold is separated from the lower mold, and then the microneedle is taken out from the first molding cavity, therefore the problem that protrusions on the microneedles are damaged during demolding is avoided.

### BRIEF DESCRIPTION OF THE DRAWINGS

FIG. 1 is a schematic diagram of a microneedle patch according to some embodiments of the present disclosure;

FIG. 2 is a schematic diagram of a microneedle having a microneedle body with a horizontal projection in a fan shape in the microneedle patch according to some embodiments of the present disclosure;

FIG. 3 is a schematic diagram of extension of tearing cracks when a substrate layer is to be torn off of the microneedle patch according to some embodiments of the present disclosure;

FIG. 4 is a front view of FIG. 2;

FIG. 5 is a top view of FIG. 2;

FIG. 6 is a dissolution state diagram after a microneedle punctures into the skin;

FIG. 7 is a schematic diagram of a microneedle of which a microneedle body is a triangular pyramid in the microneedle patch according to some embodiments of the present disclosure;

FIG. 8 is a front view of FIG. 7;

FIG. 9 is a schematic diagram of a microneedle body being a combination of a cone and a cylinder in the microneedle patch according to some embodiments of the present disclosure (four protrusions are provided on the microneedle body, and every two protrusions among the four protrusions are oppositely provided along a circumferential side wall of the microneedle body);

FIG. 10 is a top view of FIG. 9;

FIG. 11 is a schematic diagram of a microneedle body being a combination of a cone and a cylinder in the microneedle patch according to some embodiments of the present disclosure (eight protrusions are provided on the microneedle body, and every two protrusions are symmetrically provided along a circumferential side wall of the microneedle body);

FIG. 12 is a schematic diagram from another viewing angle of FIG. 11;

FIG. 13 is a schematic diagram from another viewing angle of FIG. 11;

FIG. 14 is a top view of FIG. 11;

FIG. 15 is a front view of FIG. 11;

FIG. 16 is a schematic diagram of a microneedle of which a microneedle body is a quadrangular pyramid in the microneedle patch according to some embodiments of the present disclosure;

FIG. 17 is a front view of FIG. 16;

FIG. 18 is a schematic diagram of providing a microneedle unit on a substrate layer;

FIG. 19 is a front view of FIG. 18;

FIG. 20 is a schematic diagram of extension of tearing cracks when a substrate layer is to be torn off of the microneedle patch according to some embodiments of the present disclosure (the microneedle units are arranged at intervals in multiple rows and multiple columns);

FIG. 21 is a schematic diagram of extension of tearing cracks when a substrate layer is to be torn off of the microneedle patch according to some embodiments of the present disclosure (the microneedle units are arranged in a staggered manner);

FIG. 22 is a schematic diagram of a microneedle patch in which guide ports are provided on a substrate layer;

FIG. 23 is an exploded view of a microneedle mold and a microneedle patch molded thereby according to some embodiments of the present disclosure;

FIG. 24 is a schematic diagram of an inner mold for molding a needle base and a microneedle having a microneedle body with a horizontal projection in a fan shape;

FIG. 25 is a cross-sectional view of an inner mold for molding a needle base and a microneedle having a microneedle body with a horizontal projection in a fan shape;

FIG. 26 is a schematic diagram of a microneedle mold comprising an upper mold provided with an upper mold bottom plate through-hole, and a microneedle molded by the microneedle mold;

FIG. 27 is a schematic diagram of an upper mold provided with an upper mold bottom plate through-hole;

FIG. 28 is an exploded view of a microneedle of which a microneedle body is a triangular pyramid and a microneedle mold for molding the microneedle according to some embodiments of the present disclosure;

FIG. 29 is a schematic diagram of the inner mold in FIG. 28;

FIG. 30 is an exploded view of a microneedle of which a microneedle body is a quadrangular pyramid and a microneedle mold for molding the microneedle according to some embodiments of the present disclosure;

FIG. 31 is a schematic diagram of the inner mold in FIG. 30;

FIG. 32 is an exploded view of the microneedle of FIG. 9 and a microneedle mold for molding the microneedle;

FIG. 33 is a cross-sectional view of the inner mold of FIG. 32;

FIG. 34 is a schematic diagram of an inner mold in a microneedle mold for molding the microneedle of FIG. 11;

FIG. 35 is a schematic diagram of embedding between an inner mold and a microneedle which has a needle base and a microneedle body both having a horizontal projection in a fan shape;

FIG. 36 is an exploded view of a microneedle patch of which a substrate layer is provided with a microneedle unit, and a microneedle mold for molding the microneedle patch;

FIG. 37 is a size marking plot of the inner mold of FIG. 25; and

FIG. 38 shows experimental data of puncture force when a first microneedle punctures into the skin and withdrawal force in different examples and comparative examples of a first protrusion, a second protrusion and a third protrusion.

### DESCRIPTION OF REFERENCE SIGNS

1. Upper mold; 11. Upper mold bottom plate; 12. Inner mold; 120. Inner mold body; 121. Cavity; 122. Groove; 123. Guide port mold; 1221. First groove; 1222. Second groove; 1223. Third groove; 13. Positioning insertion rod; 14. Upper mold bottom plate through-hole; 2. Middle plate; 21. Middle plate through-hole; 22. Positioning hole; 3. Lower mold; 31. First molding cavity; 32. Positioning slot; 33. Second molding cavity; 4. First microneedle; 40. Second microneedle; 41. Microneedle body; 411. Needle tip; 412. Middle part; 413. Needle base; 414. Guide port; 4141. First cutting line; 4142. Second cutting line; 42. Protrusion; 421. First protrusion; 422. Second protrusion; 423. Third protrusion; 5. Substrate layer; 51. Substrate through-hole; 6. Crack; 60. Intersection point; A. Joint point;
T0. State when a first microneedle just punctures into the skin;
T1. State at 20 minutes after the first microneedle punctures into the skin;
T2. State at 40 minutes after the first microneedle punctures into the skin;
T3. State at 60 minutes after the first microneedle punctures into the skin;
X. Direction from the side of the first microneedle provided with protrusions to the side not provided with protrusions, or an extension direction from the side of the first microneedle provided with protrusions to the side provided with a second direction in the same microneedle unit.

### DETAILED DESCRIPTION OF THE EMBODIMENTS

Hereinafter, embodiments of the present disclosure are described in combination with particular specific examples. Other advantages and effects of the present disclosure will be readily learned by a person skilled in the art from the content disclosed in the present description. The present disclosure may also be implemented or applied by other different specific embodiments, and various modifications or changes can also be made to various details of the present description on the basis of different opinions and applications without departing from the spirit of the present disclosure.

Before further describing specific embodiments of the present disclosure, it should be understood that the scope of protection of the present disclosure is not limited to the particular specific embodiments described below; and it should also be understood that the terms used in the embodiments of the present disclosure are for describing particular specific embodiments, and are not intended to limit the scope of protection of the present disclosure.

In order to stably embed microneedles into the skin, a related art includes the following two modes:
a first mode is: a substrate layer is an adhesive tape, ensuring that after microneedles puncture into the skin, the substrate layer can remain adhered to a surface of the skin, so as to achieve continuous force applying to the microneedles, and the substrate layer is torn off after the microneedles are dissolved. However, people who are sensitive to adhesive tapes cannot use this type of microneedle patches, and children, or other special patients, pets, etc. may cause fall-off of the substrate layer due to scratching, and thus the administration effect of the microneedles cannot be achieved.

A second mode is: by formulation control, an easily-broken layer easier to dissolve relative to drug-containing components is provided at the microneedle root, connected to the substrate, of each microneedle; however, the root of the microneedle close to the substrate layer is near the corneum of the skin, even if a microneedle material that is instantly soluble in water has a very low dissolving rate in the skin, and the dissolving-broken time of the easily-broken layer is long; therefore, the practical operation feasibility of making the substrate layer easy to tear off after the microneedles are dissolved is limited.

### Embodiment 1

The present embodiment provides a microneedle patch, the microneedle patch includes a substrate layer 5 and at least one microneedle; the at least one microneedle is arranged at a bottom of the substrate layer 5. The microneedle itself carries a medicine component and can be dissolved in skin.

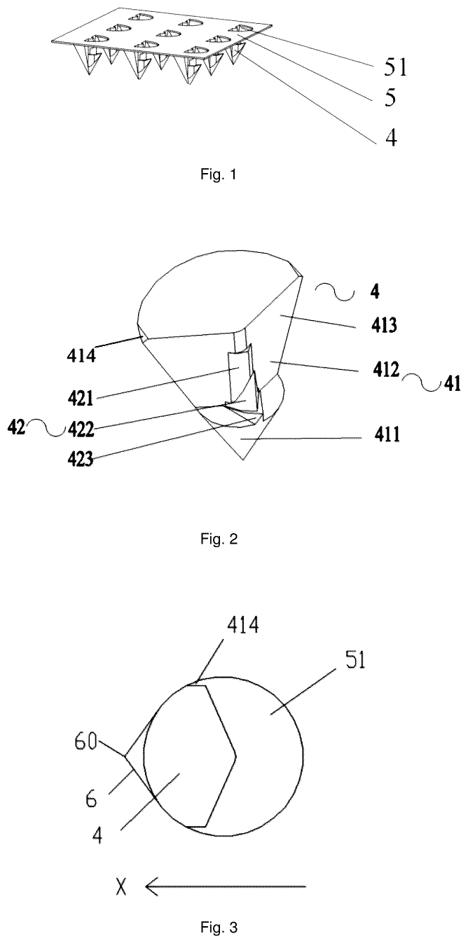

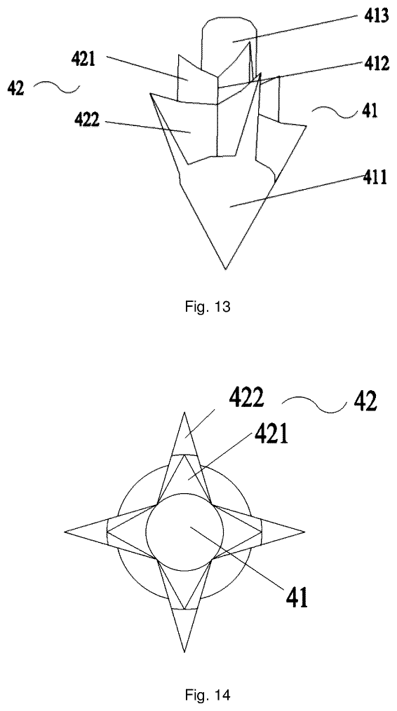

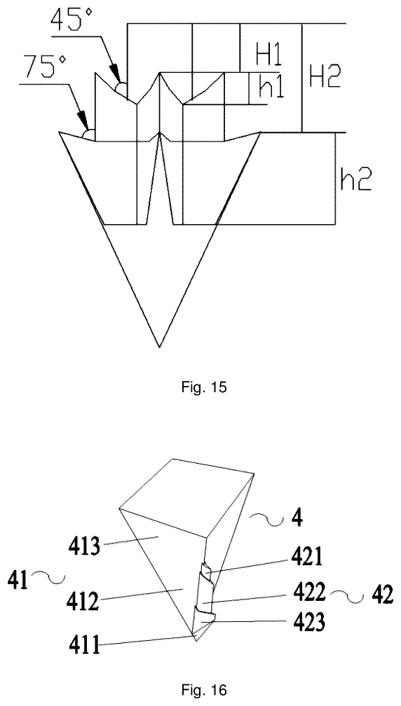

Furthermore, as shown in FIGS. 1-21, the microneedle includes a first microneedle 4, and the first microneedle 4 includes a microneedle body 41. In a vertical direction, the microneedle body 41 includes a needle tip 411, a middle part 412 and a needle base 413 in sequence. The needle base 413 is connected with the substrate layer 5. A circumference of the microneedle body 41 is provided with a fixing portion having a non-smooth surface, and the fixing portion includes a protrusion 42 and/or a part of the needle tip 411 circumferentially protruding from the needle base 413, or any form of bulge which is arranged on the microneedle body 41 and can be anchored to the skin after the microneedle punctures into the skin. In this embodiment, a circumference of the middle part 412 is provided with at least one protrusion 42. The microneedle body 41 and the protrusion 42 are integrally formed by a mold, and certainly may also be prepared by 3D printing, centrifugation forming, stretch forming, and the like. The protrusion 42 is pyramid-shaped, and the protrusion 42 extends radially along the microneedle body 41 and has a tip end away from the microneedle body 41.

When the first microneedle 4 is located in the skin, it needs to consider whether the first microneedle 4 can still remain at a desired position in the skin when the first microneedle is completely dissolved, and thus a gripping force of the first microneedle 4 for the skin is increased by the described arrangement.

In particular, the first microneedle 4 has the following advantages:

After the first microneedle 4 punctures into and enters the skin, the first microneedle 4 is anchored to the skin by the protrusion 42 and/or the part of the needle tip 411 protruding from the needle base 413, which increases the gripping force between the first microneedle 4 and the skin, and prevents the first microneedle 4 from being detached from the skin due to elastic deformation and pressing of the skin, thereby achieving continuous and accurate administration, and ensuring the administration effect.

The microneedle patch when used, after the first microneedle 4 punctures into the skin, the protrusion 42 and the part of the needle tip 411 protruding from the needle base 413 on the first microneedle 4 are embedded into the skin, to increase the gripping force between the first microneedle 4 and the skin, and thus after the first microneedle 4 punctures into the skin, when an external force is applied to tear and remove the substrate layer 5, the first microneedle 4 can still be embedded in the skin to prevent the microneedle from being dragged out of the skin, such that the substrate layer 5 can be immediately torn off from the microneedle patch, improving the comfort and aesthetics of the microneedle patch during use; especially, for situations of scratching or licking the substrate layer 5 in an administration process of a child, a pet and a psychopath, the first microneedle 4 is prevented from falling off.

In order to enhance the strength of the first microneedle 4 and reduce the resistance of the first microneedle 4 puncturing into the skin, in some embodiments, the protrusion 42 is provided in the middle part 412 of the microneedle body 41. As the protrusion 42 is located in the middle part 412 of the microneedle body 41, a weakened region of the needle tip 411 on the microneedle body 41 can be avoided, and the strength of the first microneedle 4 can be ensured, so as to avoid a problem that failure of the first microneedle 4 caused by breaking of the needle tip 411 due to insufficient strength when the first microneedle 4 punctures into the skin. On the other hand, the protrusion 42 is arranged in the middle part 412 of the microneedle body 41, a channel which guides the microneedle into the skin is formed in the process of the needle tip 411 puncturing into and entering the skin. There is no other structure on the needle tip 411, a puncture resistance is small, and deformation recovery of the skin has a certain delay; therefore, the first microneedle 4 and the protrusion 42 can be guided to enter the skin smoothly, thereby avoiding or reducing the occurrence or probability of problem of breaking of the protrusion 42 during puncturing into and entering the skin, thereby ensuring that after the first microneedle 4 completely enters the skin, the first microneedle 4 can be stably embedded in the skin due to the characteristics of skin elasticity and recovery after deformation.

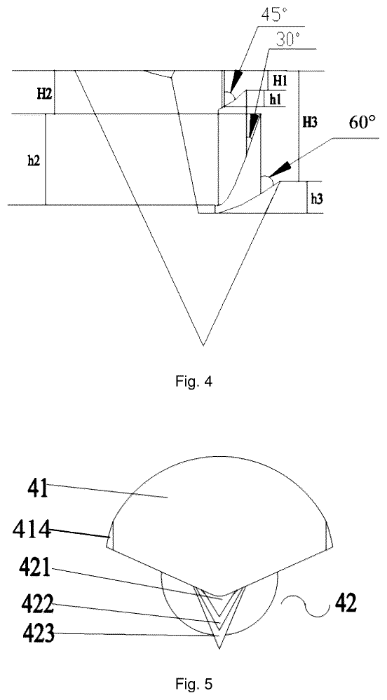

In some embodiments, the protrusion 42 extends in a direction from the needle tip 411 to the substrate layer 5 in the middle part 412, so that the protrusion 42 forms a Bee Sting Shape structure, and the first microneedle 4 can be stably anchored in the skin, thereby increasing the gripping force between the first microneedle 4 and the skin. As shown in FIGS. 2 and 4, in some embodiments, an included angle a between an extension direction of the protrusion 42 and the axis of the microneedle body 41 (a vertical line passing through the needle tip 411) is 15°≤α≤75°, more preferably, 30°≤α≤60°, such that after the first microneedle 4 punctures into the skin, the protrusion 42 can effectively puncture into the skin secondarily, and thus the skin is anchored between the microneedle body 41 and the protrusion 42, and the gripping force between the first microneedle 4 and the skin is enhanced, thereby increasing the resistance between the first microneedle 4 and the skin, and ensuring to satisfy that an embedding resistance (the gripping force) between the first microneedle 4 and the skin is greater than a peeling force by which the substrate layer 5 is broken and separated from the first microneedle 4, and preventing the first microneedle 4 from being dragged out together with the substrate layer 5 and being detached from the skin when the substrate layer 5 is torn off; or preventing the microneedle 4 from being squeezed out of the skin when an adhesive substrate layer cannot be well adhered to the skin due to excessive skin secretions after the microneedle patch is adhered to the skin, or preventing the microneedle 4 from being squeezed out of the skin after the microneedle is punctured into the skin when the substrate layer is a non-adhesive substrate layer, thereby satisfying the use requirement for microneedle 4 of people allergic to an adhesive substrate layer.

When the included angle α>75° C., the first microneedle 4 has a high resistance during puncturing into the skin and is easy to break; further, when the first microneedle 4 punctures into the skin, as the skin has both elasticity and stiffness, the first microneedle 4 cannot be well attached to the skin, the skin may be easily slide out between the microneedle body 41 and the protrusion 42, the secondary puncturing effect of the protrusion 42 into the skin is poor, and the anchoring effect of the first microneedle 4 in the skin is poor. When the included angle α<15° C., a contact area between the microneedle body 41 and the protrusion 42 and the skin is too small, the resistance between the first microneedle 4 and the skin is small, and the first microneedle easily slides out of the skin, secondary puncturing of the protrusion 42 into the skin cannot be achieved, so that the anchoring effect of the first microneedle 4 in the skin is poor.

Further, as shown in FIGS. 2-17, the microneedle body 41 may have various shapes, including a cone with a horizontal projection in a fan shape, a cone with a horizontal projection in an elliptic fan shape, a triangular pyramid, a quadrangular pyramid, and other shapes.

For better explanation, as an example, the following content relates to the specific arrangements of the protrusion 42 arranged on the microneedle body 41 of several shapes in the first microneedle 4:

As shown in FIGS. 2 and 4-6, when the horizontal projection of the needle base 413 of the first microneedle 4 is fan-shaped and the microneedle body 41 is a cone with a horizontal projection in a fan shape: the protrusion 42 is located on an edge of the microneedle body 41 connected to the tip end of the fan-shaped flat surface, and thus on the one hand, this reduces the resistance when the first microneedle 4 punctures into the skin, and on the other hand, the deformation of the skin in contact with a fan-shaped region after the first microneedle 4 punctures into the skin is large, and the skin deformation in an edge contact region connected to the tip end of the fan-shaped flat surface is small, and the skin can be well attached to the protrusion 42 on the first microneedle 4, facilitates the anchoring effect between the protrusion 42 and the skin.

In some embodiments, along the extension direction from the needle base 413 to the needle tip 411, the number of the protrusion 42 is at least one, preferably two to three, and the protrusions 42 are sequentially arranged along the edge of the microneedle body 41 connected to the tip end of the fan-shaped flat surface. If only one protrusion 42 is provided, along with the first microneedle 4 puncturing into the skin, the protrusion 42 is dissolved, and then the first microneedle 4 and the skin cannot continuously maintain a stable skin gripping force, and the first microneedle 4 is easily squeezed out of the skin. However, the greater the number of the protrusions 42, the smaller the volume of a single protrusion 42, and the easier the dissolution of the protrusions 42, thereby losing the effect of the gripping force between the protrusions and the skin.

When the number of the protrusions 42 is two or more, along the direction from the needle base 413 to the needle tip 411 of the first microneedle 4, the horizontal projection areas and volumes of the plurality of protrusions 42 sequentially increase, so as to increase and continuously realize the gripping force between the first microneedle 4 and the skin.

It should be noted that when the first microneedle 4 is provided with two protrusions 42 along the direction in which the needle base 413 extends towards the needle tip 411, the protrusions 42 sequentially include a first protrusion 421 and a second protrusion 422. The shapes of the protrusions 42 are set as not affecting demolding. Specifically:

A circumferential side wall of the first protrusion 421 extends vertically, or is obliquely arranged in a direction close to the microneedle body 41; and a circumferential side wall of the second protrusion 422 extends vertically, or abuts against a circumferential side wall of a molding cavity of a mold, so as to prevent the first protrusion 421 from affecting demolding of the second protrusion 422.

When the first microneedle 4 is provided with two or more protrusions 42 along the direction in which the needle base 413 extends towards the needle tip 411, the protrusions 42 sequentially include a first protrusion 421, a second protrusion 422 and other protrusions 42, and a third protrusion 423. The shapes of the protrusions 42 are set as not affecting demolding. Specifically:

A circumferential side wall of the first protrusion 421 extends vertically. Circumferential side walls of the second protrusion 422 and other protrusions 42 located between the first protrusion 421 and the third protrusion 423 extend vertically, or are obliquely arranged in a direction close to the microneedle body 41. A circumferential side wall of the third protrusion 423 extends vertically, or abuts against a circumferential side wall of a molding cavity of a mold. The described arrangements can prevent one protrusion 42 from affecting demolding of the other protrusion.

In some embodiments, along the extension direction of the needle base 413 towards the needle tip 411, the protrusions 42 sequentially includes a first protrusion 421, a second protrusion 422 and a third protrusion 423, and the horizontal projection areas and volumes of the first protrusion 421, the second protrusion 422 and the third protrusion 423 sequentially increase.

In some embodiments, a vertical distance H1 between a tip end of the first protrusion 421 and the substrate layer 5 is 100 μm-130 μm, and the height h1 from a base part to a pointed tip of the first protrusion 421 is 30 μm; a vertical distance H2 between a tip end of the second protrusion 422 and the substrate layer 5 is 250 μm-350 μm, and the height h2 from a base part to a pointed tip of the second protrusion 422 is 100 μm; and a vertical distance H3 between a tip end of the third protrusion 423 and the substrate layer 5 is 250 μm-450 μm, and the height h3 from a base part to a pointed tip of the third protrusion 423 is 50 μm.

As shown in FIG. 4, in some embodiments, an included angle between the extension direction of the first protrusion 421 and an axis of the microneedle body 41 (a vertical line passing through the needle tip 411) is 45°; an included angle between the extension direction of the second protrusion 422 and the axis of the microneedle body 41 (the vertical line passing through the needle tip 411) is 30°; and an included angle between the extension direction of the third protrusion 423 and the axis of the microneedle body 41 (the vertical line passing through the needle tip 411) is 60°. As the skin has elasticity and rigidity, the first microneedle 4 cannot be tightly attached to the skin, the included angle between the extension direction of the third protrusion 423 and the axis of the microneedle body 41 is 60°, the height h3 from the base part to the pointed tip is 50 μm, an gap exists between the first microneedle 4 and the skin after the first microneedle punctures into the skin, and a secondary puncturing effect between the first protrusion 421 and the skin is weak. But the included angle between the extension direction of the second protrusion 422 and the axis of the microneedle body 41 is 30°, and the height h2 from the base part to the pointed tip is 100 μm, the gap with the skin is relatively small, and the attachment effect is relatively good; when the deformation of the skin recovers and will squeeze out the first microneedle 4, the second protrusion 422 is easy to puncture into the skin secondarily, which increases the resistance between the first microneedle 4 and the skin, so that the first microneedle 4 can stay in the skin. Furthermore, after the first microneedle 4 punctures into the skin and the deformation of the skin recovers to a certain degree, the gap between the skin and the third protrusion 423 and the first protrusion 421 on the first microneedle 4 is relatively reduced, the attachment effect is relatively good, and the first microneedle can better puncture into the skin secondarily, such that the first microneedle 4 can be stably embedded in the skin. In addition, after the first microneedle 4 punctures into the skin, the first protrusion 421 is located in the corneum of the skin, the dissolution speed is relatively slow, thus the volume of the first protrusion 421 should not be too large.

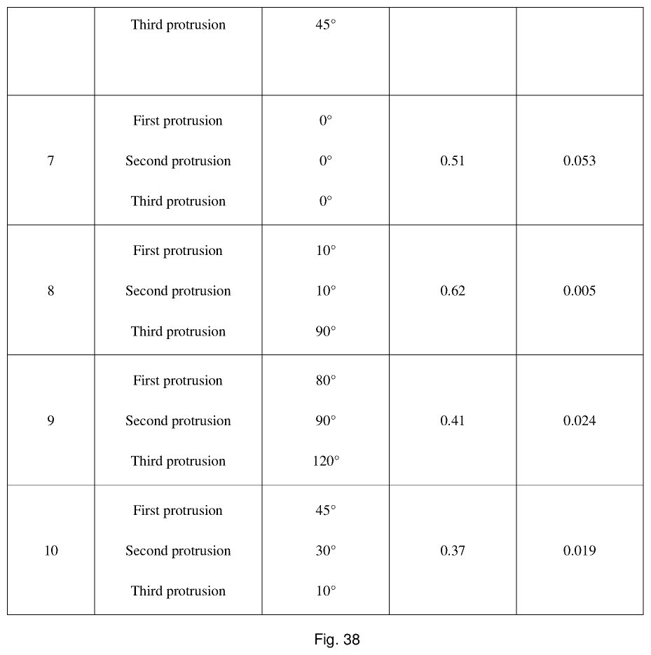

FIG. 38 shows experimental data of puncture force when the first microneedle 4 punctures into the skin and withdrawal force in different examples and comparative examples of the first protrusion 421, the second protrusion 422 and the third protrusion 423.

Specifically, a crushing force of the first microneedle 4 in some embodiments of the present disclosure, i.e. the maximum resistance value that the first microneedle 4 can bear when puncturing into the skin, is greater than or equal to 0.1 N, such that the first microneedle 4 can completely puncture into the skin smoothly. Moreover, the withdrawal force after the first microneedle 4 punctures into the skin, i.e. the gripping force between the first microneedle 4 and the skin after the first microneedle punctures into the skin, is greater than or equal to 0.06 N, and the force by which the substrate layer 5 is torn off and separated from the first microneedle 4 is less than 0.06 N, so as to ensure that when the first microneedle 4 punctures into the skin and then the substrate layer 5 is torn off and removed, the first microneedle 4 can be stably embedded in the skin.

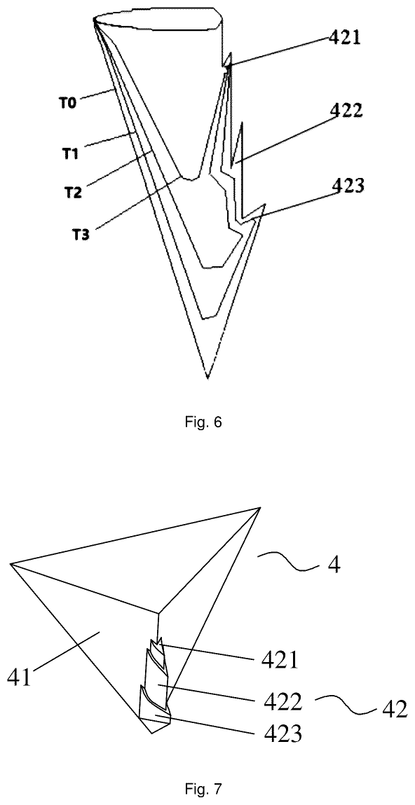

As shown in FIG. 6, by providing the above protrusions 42, after the first microneedle 4 punctures into and enters the skin, the first protrusion 421 is located in the corneum; the water content of the skin near the corneum is low, therefore, the dissolution speed of the first protrusion 421 embedded therein is relatively slow, and although the volume of the first protrusion 421 is the smallest, the first protrusion can still provide a skin gripping force for a long time. The second protrusion 422 and the third protrusion 423 are both embedded below the corneum, the skin herein has water content higher than the corneum, and the second protrusion 422 and the third protrusion 423 have a higher dissolution speed than the first protrusion 421; as the volume of the second protrusion 422 is smaller than that of the third protrusion 423, the dissolution time of the third protrusion 423 is longer than that of the second protrusion 422. At an initial stage after the first microneedle 4 enters the skin, a rebound force of the skin is the strongest, and the second protrusion 422 and the third protrusion 423 are able to provide a stronger skin gripping force to counteract the rebound force of the skin, so that the first microneedle 4 is stably embedded in the skin. As time goes on, the microneedle body 41, the second protrusion 422 and the third protrusion 423 are dissolved, the gripping force of the first microneedle 4 on the skin is reduced, and at this time, the deformation amount of the skin is reduced and the rebound force is reduced. When the second protrusion 422 is substantially completely dissolved and the third protrusion 423 is not completely dissolved, a certain gripping force can still be generated, and the dissolution degree of the first protrusion 421 is relatively small; the first protrusion 421 and the third protrusion 423 can still provide a sufficient skin gripping force, so as to ensure that the first microneedle 4 is stably embedded in the skin and is prevented from being squeezed out of the skin.

### Embodiment 2

This embodiment differs from Embodiment 1 in that: the microneedle body 41 is a cone with a horizontal projection in an elliptic fan shape, the protrusion 42 is located on an edge of the microneedle body 41 connected to a tip end of a elliptic fan-shaped bottom face, and is located on a short axis side of the horizontal projection of the microneedle body 41.

Compared with Embodiment 1, the volume of the first microneedle 4 of the present embodiment is increased, so as to increase the medicine loading capacity of the first microneedle 4, and satisfy the requirements of different medicines for dosage forms.

### Embodiment 3

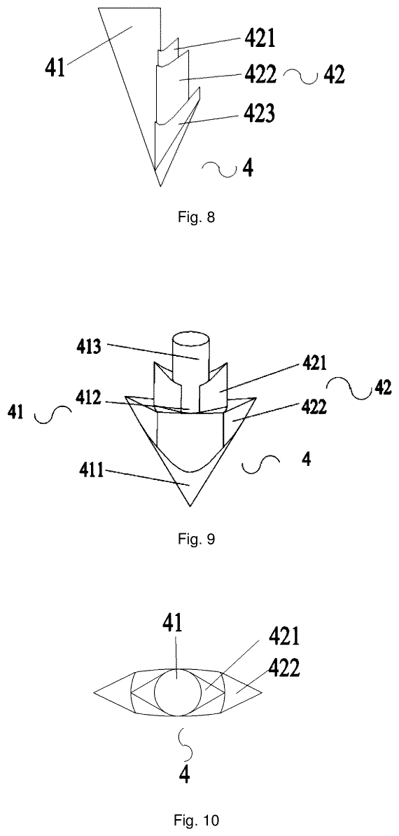

As shown in FIGS. 7-8, this embodiment differs from Embodiment 1 in that: the microneedle body 41 is of a triangular pyramid, the protrusion 42 is located on an edge of the microneedle body 41, so as to reduce the resistance when the first microneedle 4 punctures into the skin. When one protrusion 42 is provided, the protrusion 42 is located on an edge perpendicular to the substrate layer 5, so as to further reduce the resistance when the first microneedle 4 punctures into the skin.

### Embodiment 4

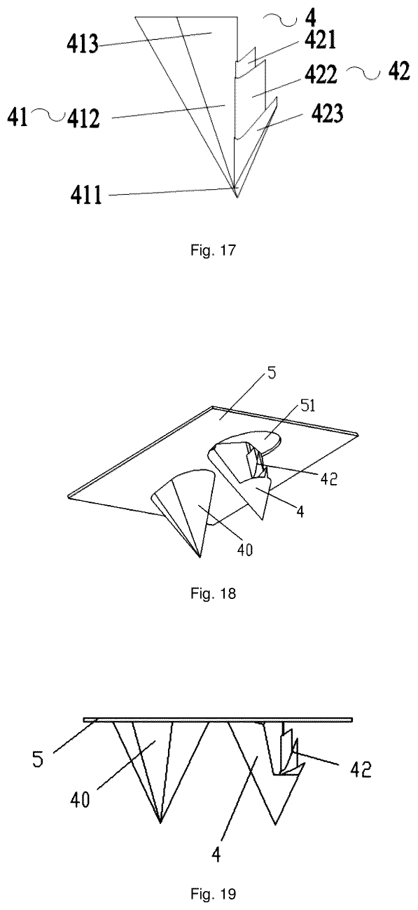

As shown in FIGS. 16-17, this embodiment differs from Embodiment 1 in that: the microneedle body 41 is of a rectangular pyramid, the protrusion 42 is located on an edge of the microneedle body 41, so as to reduce the resistance when the first microneedle 4 punctures into the skin. When one protrusion 42 is provided, the protrusion 42 is located on an edge perpendicular to the substrate layer 5, so as to further reduce the resistance when the first microneedle 4 punctures into the skin.

### Embodiment 5

As shown in FIGS. 9-10, this embodiment differs from Embodiment 1 in that: the microneedle body 41 is a combination of a cone and a cylinder; the needle tip 411 of the microneedle body 41 is a cone, and the middle part 412 and the needle base 413 are a cylinder. The number of the protrusions 42 of the first microneedle 4 is four, and in some embodiments, every two protrusions 42 among the four protrusions are respectively oppositely provided along a circumferential side wall of the middle part 412 of the microneedle body 41.

As shown in FIGS. 9-10, when the number of the protrusions 42 is four, and every two protrusions 42 among the four protrusions are respectively oppositely provided along a circumferential side wall of the middle part 412 of the microneedle body 41, along the direction from the needle base 413 to the needle tip 411 of the first microneedle 4, the horizontal projection areas and volumes of two adjacent protrusions 42 increase, so as to increase and continuously realize the gripping force between the first microneedle 4 and the skin.

In some embodiments, the protrusions 42 arranged on the circumferential side wall of the middle part 412 of the microneedle body 41 are symmetrically arranged, and are respectively first protrusions 421 and second protrusions 422; along the direction from the needle base 413 to the needle tip 411 of the first microneedle 4, the horizontal projection area and volume of the first protrusion 421 are less than those of the second protrusion 422.

In some embodiments, a vertical distance H1 between the tip end of the first protrusion 421 and the substrate layer is 50 μm, and the height h1 from the base part to the pointed tip of the first protrusion 421 is 50 μm; and a vertical distance H2 between the tip end of the second protrusion 422 and the substrate layer is 200 μm, and the height h2 from the base part to the pointed tip of the second protrusion 422 is 30 μm. An included angle α1 between the extension direction of the first protrusion 421 and the axis of the microneedle body 41 (a vertical line passing through the needle tip 411) is 45°; and an included angle α2 between the extension direction of the second protrusion 422 and the axis of the microneedle body 41 is 75°. In this embodiment, the first protrusions 421 are symmetrically arranged on the circumferential side wall of the middle part 412 of the microneedle body 41, and the second protrusions 422 are symmetrically arranged; after the first microneedle 4 punctures into the skin and stays in the skin, due to good elasticity of the skin, the first microneedle 4 punctures through the corneum under the action of an external force. When the deformation of the skin recovers, a secondary puncturing effect of the protrusions 42 in the skin is good, the anchoring effect of the first microneedle 4 is strong, and the first microneedle can be stably embedded in the skin, and thus the first microneedle 4 can be made relatively short, preventing the first microneedle 4 from puncturing into a dermis layer, achieving painless administration. In the embodiment, on the other hand, since the first microneedle 4 is a combination of a cone and a cylinder, the structure close to the needle base 413 is a cylinder, and the volume is relatively small, when the first microneedle 4 punctures into the skin, the substrate layer can be torn off immediately, so that a main body of the cone part of the first microneedle 4 is completely embedded in the skin, and effective absorption of components in the first microneedle 4 is realized, thereby achieving precise administration.

In addition, since human skin is anisotropic, for embodiment, human trunk skin, the transverse and longitudinal elasticities are significantly different, in the transverse direction, the elasticity is weak but the stress is large, and in the longitudinal direction, the elasticity is strong but the stress is small, and the transverse stress value can be almost double that of the longitudinal stress value. The larger stress of the skin can provide support for the puncturing of the first microneedle 4, so as to facilitate puncturing of the first microneedle 4. Regarding the first microneedle 4 in this embodiment, as the protrusions 42 are arranged on both sides of the microneedle body 41 and the microneedle body 41 is flat, when puncturing into the skin, the first microneedle 4 performs puncturing along a long axis direction of the first microneedle 4 and the extension direction of the trunk or limbs. Because the skin stress in the extension direction of the trunk or limbs is large, regarding the flat first microneedle 4 in this embodiment, the first microneedle 4 can puncture into the skin under a small external force, reducing the pain sensation of the skin.

### Embodiment 6

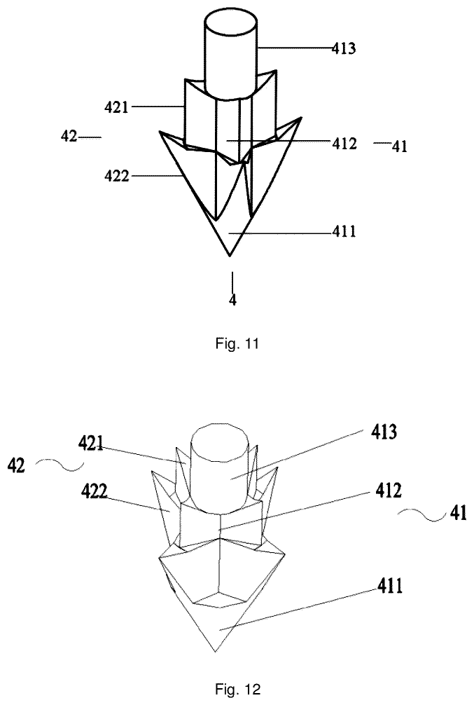

As shown in FIGS. 11-15, this embodiment differs from Embodiment 5 in that: the microneedle body 41 is a combination of a cone and a cylinder; the needle tip 411 of the microneedle body 41 is a cone, the middle part 412 and the needle base 413 are a cylinder, and the number of the protrusions 42 of the first microneedle 4 is eight. In some embodiments, four groups of protrusions 42 are symmetrically arranged along the circumferential side wall of the middle part 412 of the microneedle body 41, and each group of protrusions 42 includes two protrusions 42 respectively arranged along the direction of the axis of the microneedle body 41 (the vertical line passing through the needle tip 41).

As shown in FIGS. 11-15, along the direction from the needle base 413 to the needle tip 411 of the first microneedle 4, each group includes two protrusions 42, and the horizontal projection areas and volumes of two adjacent protrusions 42 increase in sequence, so as to increase and continuously realize the gripping force between the first microneedle 4 and the skin. Along the direction from the needle base 413 to the needle tip 411 of the first microneedle 4, two protrusions 42 arranged in each group are a first protrusion 421 and a second protrusion 422 respectively; and along the direction from the needle base 413 to the needle tip 411 of the first microneedle 4, the horizontal projection area and volume of the first protrusion 421 are less than those of the second protrusion 422.

In some embodiments, a vertical distance H1 between the tip end of the first protrusion 421 and the substrate layer is 45 μm, and the height h1 from the base part to the pointed tip of the first protrusion 421 is 30 μm; and a vertical distance H2 between the tip end of the second protrusion 422 and the substrate layer is 100 μm, and the height h2 from the base part to the pointed tip of the second protrusion 422 is 100 μm. An included angle α1 between the extension direction of the first protrusion 421 and the axis of the microneedle body 41 (a vertical line passing through the needle tip 411) is 45°; and an included angle α2 between the extension direction of the second protrusion 422 and the axis of the microneedle body 41 is 75°. In this embodiment, four groups of protrusions 42 symmetrically arranged on the circumferential side wall of the middle part 412 of the microneedle body 41, each group of protrusions 42 includes first protrusions 421 and second protrusions 422; after the first microneedle 4 punctures into the skin, due to high elasticity of the skin, the first microneedle 4 is pressed into the skin under the action of an external force and punctures through the corneum, and after the action of the external force is released, skin deformation recovers. However, the first microneedle 4 has four groups, i.e. a total of eight protrusions 42, arranged along the circumference of the microneedle body 41, a secondary puncturing effect of the protrusions 42 in the skin is good, the anchoring effect of the first microneedle 4 is strong, and the first microneedle can be stably embedded in the skin, and thus the first microneedle 4 can be made relatively short, preventing the first microneedle 4 from puncturing into a dermis layer, achieving painless administration, being more suitable for easily-escaping people such as children and pets. In the embodiment, since the first microneedle 4 is a combination of a cone and a cylinder, the structure close to the needle base 413 is a cylinder, and the volume is relatively small, when the first microneedle 4 punctures into the skin, the substrate layer can be torn off immediately, so that the main body of the cone part of the first microneedle 4 is completely embedded in the skin, and effective absorption of components in the first microneedle 4 is realized, thereby achieving precise administration.

### Embodiment 7

As shown in FIGS. 18 and 19, this embodiment differs from Embodiment 1 in that: the microneedle patch is provided with a stress point structure at a connection position between the substrate layer 5 and the first microneedle 4. In this embodiment, the stress point structure includes a substrate through-hole 51 provided on the substrate layer 5 and on the same side as the protrusion 42 on the first microneedle 4. At the plane where the substrate layer is located, along a direction parallel to the connecting line of two connecting points between the stress point structure and the first microneedle 4, the maximum length of the substrate through-hole 51 is greater than the maximum length of the first microneedle 4, so that when the substrate layer 5 is torn off, a tear-off crack can extend around two sides of the first microneedle 4 and then intersect. In some embodiments, the substrate through-hole 51 is a major arc, and the projection of the first microneedle 4 on the substrate layer 5 is a minor arc.

As shown in FIGS. 1-21, when the microneedle patch is used, after the first microneedle 4 punctures into the skin, a hand grips the substrate layer 5, and peels off the substrate layer 5 along a direction from the side of the first microneedle 4 provided with the protrusion 42 to the side not provided with the protrusion 42. There is large stress concentration at the stress point structure formed at the connection position between the substrate through-hole 51 and the needle base 413 of the first microneedle 4, when the substrate layer 5 is to be torn off, a critical stress of breaking and separating the substrate layer 5 from the first microneedle 4 at the stress point structure is small, and therefore under the action of a small external force, the substrate layer 5 is broken and separated from the first microneedle 4, so as to tear off the substrate layer 5. In order to improve the yield of the first microneedle 4 during molding, when a molding liquid is injected into a mold to mold the microneedle patch, demolding is performed from the side of the first microneedle 4 not provided with the protrusion 42 to the side of the first microneedle 4 provided with the protrusion 42, so that the microneedle patch is separated from the mold, thereby ensuring that the first microneedle 4 is complete and undamaged.

In the process of tearing off the substrate layer 5, since the first microneedle 4 is provided with the protrusion 42, the first microneedle 4 is gripped in the skin by the protrusion 42, satisfying that an embedding resistance (gripping force) between the first microneedle 4 and the skin is greater than a peeling force by which the substrate layer 5 is broken and separated from the first microneedle 4, thereby the first microneedle 4 stable embedding in the skin. In addition, along a direction parallel to the connecting line of two connecting points between the stress point structure of the first microneedle 4, the maximum size of the substrate through-hole 51 is greater than the maximum length of the first microneedle 4, so that a tear-off crack generated after the substrate layer 5 is broken and separated from the first microneedle 4 at the stress point structure can extend around two sides of the first microneedle 4 and then intersect; in this way, the substrate layer 5 can be completely separated from the first microneedle 4, and the ease of tearing of the substrate layer 5 is improved. In the present disclosure, after the first microneedle 4 punctures into the skin, the substrate layer 5 can be immediately torn off, and only the first microneedle 4 remains in the skin, further improving the comfort and aesthetics of the microneedle patch during use, which avoids carried out of the first microneedle 4 from the skin caused by situations of scratching or licking the substrate layer 5 in an administration process of a child, a pet and a psychopath.

### Embodiment 8

As shown in FIGS. 2, 3 and 5, this embodiment differs from Embodiment 1 in that: the microneedle patch is provided with a stress point structure at a connection position between the substrate layer 5 and the first microneedle 4. The stress point structure includes a substrate through-hole 51 arranged on the substrate layer 5 and guide ports 414 arranged on the needle base 413 of the first microneedle 4. The guide ports 414 are located at junctions of both sides of the needle base 413 and the substrate through-hole 51, and the guide ports 414 are located on the circumferential side wall of the needle base 413. The first microneedle 4 and the substrate through-hole 51 adjacent thereto form a group of stress unit, and at least one group of stress unit is arranged at intervals on the substrate layer 5. As shown in FIG. 3, when the first microneedle 4 punctures into the skin and the substrate layer 5 is torn and removed along the X direction, an external tearing force acts on the substrate layer 5 and is transmitted to the guide ports 414 through the substrate layer 5. The guide ports 414 reduce a critical separation force between the substrate layer 5 and the needle base 413 of the first microneedle 4, and when the microneedle patch is used and the substrate layer 5 is torn off, the guide ports 414 are able to guide tearing cracks between the substrate layer 5 and the first microneedle 4 to extend along cracks 6. Since the protrusion 42 provided on the first microneedle 4 enables the first microneedle 4 to be stably embedded in the skin, and in the process of tearing off the substrate layer 5, under a resultant force of an external force and the embedding force between the first microneedle 4 and the skin, the cracks 6 generated between the substrate layer 5 and the first microneedle 4 can precisely extend along the circumferential side wall of the needle base 413 of the first microneedle 4 by the guide ports 414, and intersect at the substrate layer 5 between two adjacent groups of stress units to form an intersection point 60, so that the first microneedle 4 is completely separated from the substrate layer 5.

### Embodiment 9

This embodiment differs from Embodiment 8 in that: the microneedle patch includes at least one microneedle unit, and each microneedle unit includes at least one first microneedle 4 and at least one second microneedle 40 which are arranged at an interval, as shown in FIGS. 18-21. The first microneedle 4 is a microneedle provided with a protrusion 42, and the second microneedle 40 is a microneedle not provided with a protrusion 42. In the microneedle unit of the present embodiment, the second microneedle 40 not provided with the protrusion 42 is able to increase the medicine loading capacity of the microneedle patch, and the first microneedle 4 provided with the protrusion 42 is able to increase the gripping force between same and the skin, thereby satisfying the requirements of a large medicine loading capacity and stable gripping with the skin of the microneedle patch.

Specifically, on the substrate layer 5, the second microneedle 40 is located at the side of the first microneedle 4 not provided with the protrusion 42, and the minimum length of the connecting line of the guide ports 414 at the two sides of the first microneedle 4 is greater than the maximum length of the second microneedle 40 along the direction parallel to the connecting line of the two guide ports 414. The guide ports 414 are located on the circumferential side wall of the needle base 413 of the first microneedle 4, and extension lines of the guide ports 414 on two sides of the first microneedle 4 intersect or are tangent to the circumferential side wall of the needle base 413 of the second microneedle 40. In some embodiments, extension lines of the guide ports 414 on two sides of the first microneedle 4 are tangent to the circumferential side wall of the needle base 413 of the second microneedle 40. In some embodiments, the microneedle body 41 is set to have a horizontal projection in a fan shape, the joint points between the guide ports 414 on both sides of the needle base 413 and the needle base 413 are A, an included angle of connecting lines respectively between the two joint points A and the center of circle of the first microneedle 4 is 120°-150°, the radius of the first microneedle 4 is R, and the radius of the second microneedle 40 is r, then 0.17R≤r≤0.59R.

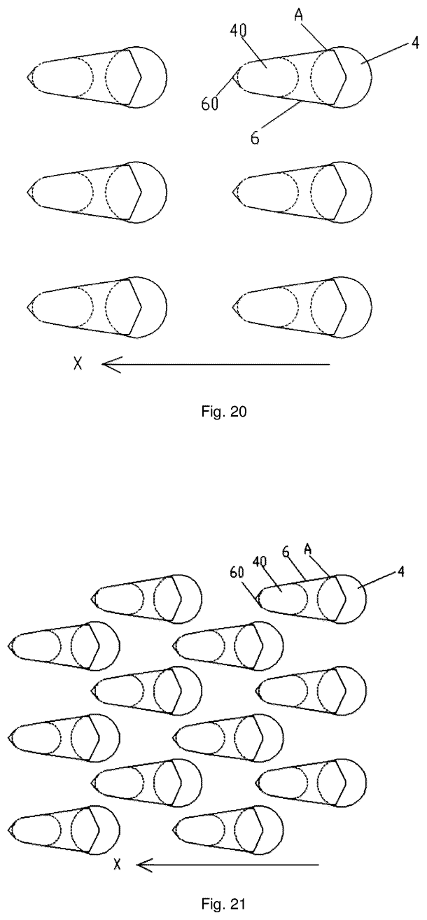

When the microneedle patch is provided with a plurality of microneedle units, the plurality of microneedle units are arranged on the substrate layer 5 at intervals in multiple rows and multiple columns, and the protrusions 42 of the first microneedles 4 extend in the same direction, as shown in FIG. 20. After the microneedle punctures into the skin, the substrate layer 5 is torn off in X direction, which is the direction from the first microneedle 4 to the second microneedle 40 of the same unit, along the side of the first microneedle 4 provided with the protrusion 42; and the cracks 6 extend along the extension directions of the joint points A, intersect at a gap between the microneedle unit and the adjacent, and form an intersection point 60. In the direction of a connecting line between the center of circle of the first microneedle 4 and the center of circle of the second microneedle 40, the distance from the center of circle of the first microneedle 4 of the microneedle unit to the center of circle of the first microneedle 4 of an adjacent microneedle unit is L, where 3R≤L≤6R, so as to ensure that the intersection point 60 formed by the cracks 6 is located between two microneedle units, and the substrate layer 5 can be effectively torn off, and the microneedle unit is stably embedded in the skin without being dragged by the substrate layer 5 and leaving the skin, thereby realizing effective and accurate administration. In addition, the substrate layer 5 has sufficient strength, after the microneedle patch is applied to the skin and the microneedles puncture into the skin, when the substrate layer 5 is torn off, a needle-free region (i.e. region not provided with the microneedle) of the substrate layer 5 has sufficient strength such that the substrate layer 5 can be torn off once, which can effectively prevent the problem that substrate layer 5 needs to be torn off for multiple times due to the breaking of the needle-free region of the substrate layer 5.

Further, when the microneedles are prepared, the amount of a raw material liquid injected into the substrate layer 5 is controlled, so that after the second microneedle 40 dries, due to drying and shrinking of the raw material liquid, the thickness of the substrate layer 5 at the connection position with the needle base 413 is less than the thickness of the substrate layer 5 in the needle-free region. Thus, after the microneedles puncture into the skin, when the substrate layer 5 is torn off, although the second microneedle 40 is not provided with the protrusion 42, as the first microneedle 4 is provided with the protrusion 42, the first microneedle 4 is stably embedded in the skin; and cracks 6 generated when the substrate layer 5 is torn off surrounds the microneedle unit and intersect at a gap between the microneedle unit and a microneedle unit adjacent thereto, such that the second microneedle 40 can be stably embedded in the skin. Moreover, as the thickness of the substrate layer 5 at the position connected to the second microneedle 40 is relatively small, the substrate layer 5 can be separated along the circumferential side wall of the second microneedle 40, and finally the microneedle units are stably embedded in the skin, achieving effective and precise administration.

When the first microneedle 4 or the microneedle unit is provided on the microneedle patch, the gripping force between the first microneedle 4 or the microneedle unit and the skin is improved by the protrusion 42 on the first microneedle 4, preventing the first microneedles 4 or the second microneedle 40 from being dragged out by the substrate layer 5 after the microneedle patch is attached to the skin and the substrate layer 5 is torn off immediately, greatly shortening the time required for the substrate layer 5 being attached to the skin when the microneedle patch is used, further improving the use comfort of the microneedle patch, facilitating use by people sensitive to the substrate layer 5 or use by children.

### Embodiment 10

This embodiment differs from Embodiment 9 in that: the microneedle units are arranged in a staggered manner, as shown in FIG. 21, the density of the microneedle units in the microneedle patch is increased, and the medicine loading capacity of the microneedle patch is increased.

### Embodiment 11

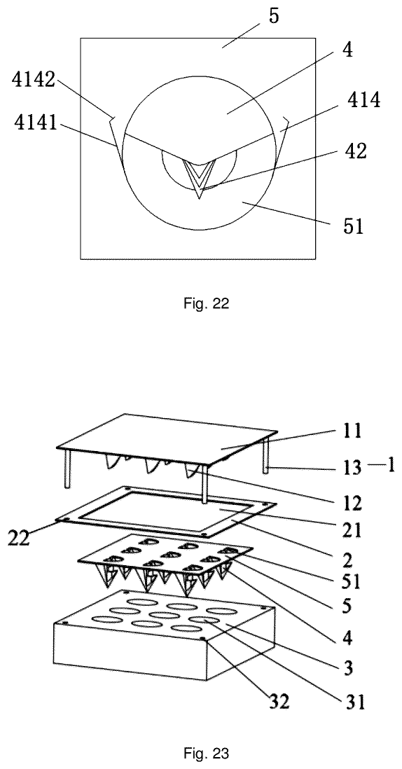

As shown in FIG. 22, this embodiment differs from Embodiment 1 in that: the microneedle patch is provided with a stress point structure at a connection position between the substrate layer 5 and the first microneedle 4. The stress point structure includes a substrate through-hole 51 arranged on the substrate layer 5 and guide ports 414 arranged at the junctions between the needle base 413 of the first microneedle 4 and the substrate layer 5, and the guide ports 414 are located on the substrate layer 5. First cutting lines 4141 and second cutting lines 4142 which are in communication with the substrate through-hole 51 are provided on the substrate layer 5 near a connection position between the substrate through-hole 51 and the needle base 413, and the guide ports 414 are formed in substrate regions surrounded by the substrate through-hole 51, the first cutting lines 4141 and the second cutting lines 4142. One end of each first cutting line 4141 is in communication with the substrate through-hole 51, and the other end is in communication with the second cutting line 4142. The second cutting line 4142 forms a certain included angle with the first cutting line 4141, so that the extension lines of the second cutting lines 4142 on two sides of the needle base 413 are able to intersect at the substrate layer 5 between the stress unit and an adjacent stress unit.

When the first microneedle 4 of the microneedle patch punctures into the skin and the substrate layer 5 is torn and removed along the X direction, an external tearing force acts on the substrate layer 5 and is transmitted to the guide ports 414 through the substrate layer 5. The guide ports 414 reduce a critical separation force between the substrate layer 5 and the needle base 413 of the first microneedle 4, and when the microneedle patch is used and the substrate layer 5 is torn off, the guide ports 414 can guide tearing cracks between the substrate layer 5 and the first microneedle 4 to extend along the second cutting lines 4142 and to intersect at the substrate layer of two adjacent stress units.

### Embodiment 12

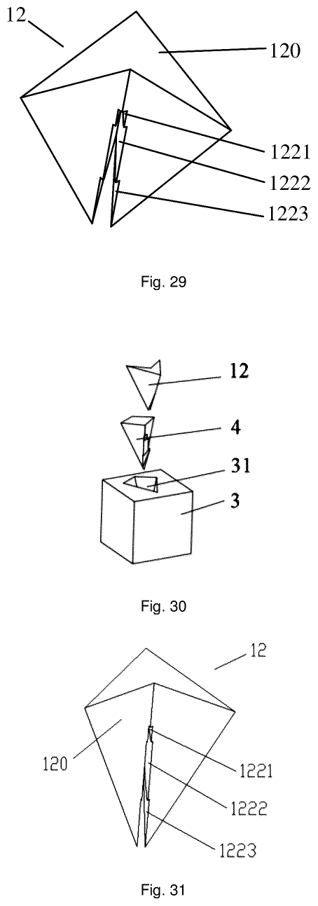

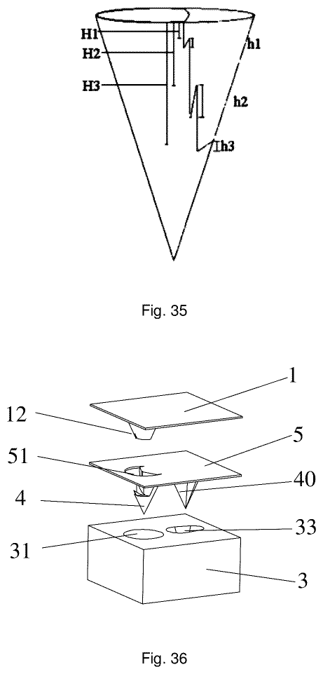

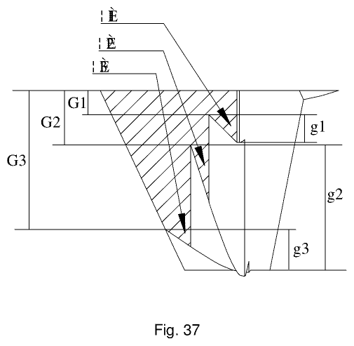

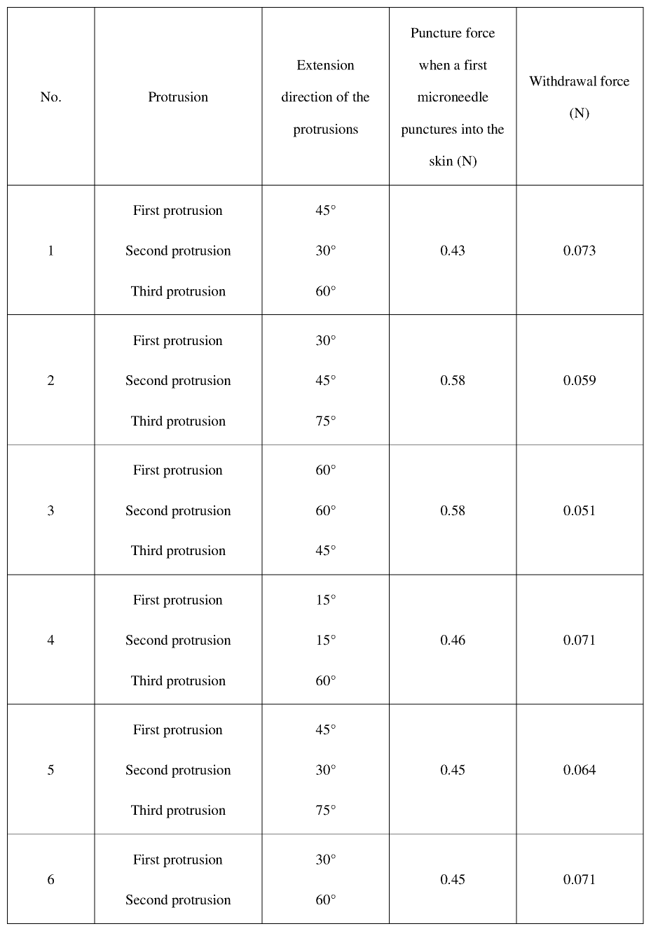

As shown in FIGS. 23-37, this embodiment further provides a microneedle mold for manufacturing the microneedle patch above.

The microneedle mold in this embodiment sequentially includes an upper mold 1 and a lower mold 3 from top to bottom, wherein the upper mold 1 is detachably connected to the lower mold 3. The upper mold 1 includes an upper mold bottom plate 11, and at least one inner mold 12 is arranged at a bottom of the upper mold bottom plate 11. At least one groove 122 is provided on the inner mold 12, so as to form at least one protrusion 42 on a middle part 412 of a first microneedle 4.

Due to the arrangement of the inner mold 12, the molded first microneedle 4 having a non-smooth surface can be easily taken out from the microneedle mold.

Further, the groove 122 may be provided inside the inner mold 12, so that the protrusion 42 can be molded on the microneedle, thereby improving the gripping force against the skin during use.

The top of the lower mold 3 is provided with at least one first molding cavity 31. Each first molding cavity 31 is provided corresponding to the at least one inner mold 12, and the first molding cavity 31 accommodates the inner mold 12. A space enclosed by the first molding cavity 31 and the inner mold 12 is used for molding the first microneedle 4.

By providing the groove 122 on the inner mold 12, to form at least one protrusion 42 on the middle part 412 of the first microneedle 4, after the first microneedle 4 is molded, first the upper mold 1 is separated from the lower mold 3, and then the first microneedle 4 is taken out from the first molding cavity 31, so as to prevent the protrusion 42 or the part of a needle tip 411 protruding from the middle part 412 on the first microneedle 4 from being damaged during demolding.

FIG. 25 is a cross-sectional view of the inner mold. Specifically, the inner mold 12 includes an inner mold body 120, the groove 122 provided on the inner mold body 120, and guide port molds 123. The guide port molds 123 are protuberances extending in the circumferential direction and provided on a maximum diameter end face of the inner mold body 120. The length of a connecting line between two ends of open ends of the maximum diameter end face of the inner mold body 120 having the guide port molds 123 is greater than the length of a connecting line between joint points of the two guide ports 414 on the first microneedle 4 and the outer circumferential side wall of the needle base 413. In some embodiments, the maximum diameter end face of the inner mold body 120 is a major arc, and the maximum diameter end face of the first microneedle 4 is a minor arc, so that when a substrate layer 5 of the microneedle patch is torn off, tearing cracks can extend around two sides of the first microneedle 4 and then intersect. A direction from the smaller diameter end to the larger diameter end of the inner mold body 120 is a Y direction, and the groove 122 extends along the Y direction. An included angle between an extension direction of the groove 122 and an axis of the inner mold 12 is θ, in some embodiments, 15°≤θ≤75°, and more preferably 30°≤θ≤60°.

Further, as shown in FIG. 25, the number of the groove 122 is at least one, and along a direction opposite to the Y direction, the volumes of the grooves 122 increase sequentially, so that the volumes of corresponding protrusions 42 in the molded first microneedle 4 increase sequentially.

In some embodiments, the number of the grooves 122 is at least two, more preferably three, which are respectively a first groove 1221, a second groove 1222, and a third groove 1223.

In some embodiments, a distance G1 between the bottom of the first groove 1221 and a large diameter end face of the inner mold 12 is 100 μm-130 μm, and a vertical extension distance g1 of the first groove 1221 is 30 μm; a distance G2 between the bottom of the second groove 1222 and the large diameter end face of the inner mold 12 is 250 μm-350 μm, and a vertical extension distance g2 of the second groove 1222 is 100 μm; and a distance G3 between the bottom of the third groove 1223 and the large diameter end face of the inner mold 12 is 250 mm-450 mm, and a vertical extension distance g3 of the third groove 1223 is 50 mm. An included angle θ1 between the extension direction of the first groove 1221 and the axis of the inner mold 12 is 45°, an included angle θ2 between the extension direction of the second groove 1222 and the axis of the inner mold 12 is 30°, and an included angle θ3 between the extension direction of the third groove 1223 and the axis of the inner mold 12 is 60°. Along the Y direction, a cross section of the grooves 122 provided in the inner mold body 120 is a vertical wall having a pattern draft, to facilitate pull-out of the inner mold 12 from the first molding cavity 31 when preparing the first microneedle 4, ensuring that the protrusions 42 are complete and undamaged, and ensuring the yield of products.

When the groove 122 includes the first groove 1221 and the second groove 1222; the first groove 1221 is used for molding a first protrusion 421 on the first microneedle 4, and the second groove 1222 is used for molding a second protrusion 422, so as to mold two protrusions 42 in the Y direction of the first microneedle 4; on this basis, a third protrusion 423 is formed between the first molding cavity 31 and an end part of the inner mold body 120 close to the first molding cavity 31, so as to form three protrusions 42 in the Y direction of the first microneedle 4.

When the groove 122 includes the first groove 1221, the second groove 1222 and the third groove 1223: the first groove 1221 is used for molding a first protrusion 421, the second groove 1222 is used for molding a second protrusion 422, and the third groove 1223 is used for molding a third protrusion 423, so as to mold three protrusions 42 in the Y direction of the first microneedle 4. Certainly, a fourth protrusion may also be formed between the first molding cavity 31 and an end part of the end of the inner mold body 120 close to the first molding cavity 31.

Further, for better explanation, as an embodiment, the following relates to a specific structure of a microneedle mold for molding the first microneedle 4 of the shape as shown in FIG. 2 in Embodiment 1:

When the microneedle body 41 is a cone with a horizontal projection in a fan shape, the first molding cavity 31 of the lower mold 3 is conical.

When the microneedle body 41 is a cone with a horizontal projection in an elliptic fan shape, the first molding cavity 31 of the lower mold 3 is a cone with a horizontal projection in an elliptic shape.

When the microneedle body 41 is a triangular pyramid, the first molding cavity 31 of the lower mold 3 is a triangular pyramid.

When the microneedle body 41 is a quadrangular pyramid, the first molding cavity 31 of the lower mold 3 is a quadrangular pyramid.

When the microneedle body 41 is a combination of a cone and a cylinder, the horizontal projection of the inner mold 12 is circular or elliptical. When the horizontal projection of the inner mold 12 is circular, the first molding cavity 31 of the lower mold 3 is conical. When the horizontal projection of the inner mold 12 is elliptical, the first molding cavity 31 of the lower mold 3 is elliptic cone-shaped.

In some embodiments, the lower mold 3 is injection molded by polydimethylsiloxane, and the thickness of the lower mold 3 is 5000 μm. The first molding cavity 31 of the lower mold 3 is treated by a Teflon spray coating process, thereby preventing the first molding cavity 31 from being adhered to the first microneedle 4, so as to facilitate the separation of the first microneedle 4 from the first molding cavity 31.

Further, in order to facilitate combination of the upper mold 1 and the lower mold 3, at least one positioning insertion rod 13 is provided at the bottom of the upper mold 1, and at least one positioning slot 32 correspondingly adapted to the at least one positioning insertion rod 13 is provided at the top of the lower mold 3.

When preparing the microneedle patch, by pre-positioning between the positioning insertion rod 13 of the upper mold 1 and the positioning slot 32 of the lower mold 3, it is ensured that each inner mold 12 of the upper mold 1 corresponds to each first molding cavity 31 of the lower mold 3, thereby improving a positioning precision of the inner molds 12 and the first molding cavities 31, avoiding a problem that the lower mold 3 is damaged due to positioning deviation when the upper mold 1 and the lower mold 3 are aligned and engaged, thereby affects the preparation precision of the first microneedles 4.

In some embodiments, the end of each positioning insertion rod 13 close to the upper mold 1 is cylindrical, and the other end is conical, to facilitate insertion of the positioning insertion rod 13 into the positioning slot 32. The shape of the positioning slot 32 matches the shape of the positioning insertion rod 13, so that the positioning insertion rod 13 and the positioning slot 32 can be precisely and steadily matched. The provision of the positioning insertion rods 13 and the positioning slots 32 prevents the upper mold 1 and the lower mold 3 from being staggered under the action of an external force in the process of preparing the first microneedles 4, which further changes the shape of the spaces enclosed by the inner molds 12 and the first molding cavities 31, causing low preparation precision of the first microneedles 4.

In some embodiments, the height of the positioning insertion rod 13 is 600 μm, and the diameter of the end of a conical shape is 300 μm.

In some embodiments, four positioning insertion rods 13 and four positioning slots 32 are provided, the four positioning insertion rods 13 are respectively located at four end points of the upper mold bottom plate 11, and the four positioning slots 32 are respectively located at four end points of the top of the lower mold 3, so that the upper mold 1 is stably combined on the lower mold 3.

In some embodiments, a preparation method for a microneedle patch is provided, the preparation method includes the following two modes:
when the molded substrate layer 5 is a complete plate body, a two-step molding method is used, i. e. first molding the first microneedles 4, and then preparing same into a microneedle patch. The method includes the following steps:
A1: combining an upper mold 1 and a lower mold 3, inserting the inner mold 12 into the first molding cavity 31, and injecting a formulation liquid into spaces enclosed by the first molding cavity 31 and the inner mold 12; and
A2: drying and molding, separating the upper mold 1 from the lower mold 3, the substrate layer 5 being attached to the first microneedles 4, and plugging out the first microneedles 4.

When the molded substrate layer 5 is a hollow plate body, i.e. the substrate layer 5 is provided with a substrate through-hole 51, an integral forming method is used, that is, the first microneedles 4 and the first substrate layer 5 are integrally formed into a microneedle patch. The method includes the following steps:
B1: filling a formulation liquid into the first molding cavity 31, then combining an upper mold 1 and a lower mold 3, and inserting an inner mold 12 into the first molding cavity 31; and
B2: drying and molding, separating the upper mold 1 from the lower mold 3, and plugging out the molded first microneedle 4.

Specifically, the drying conditions in both steps A2 and B2 are: drying for 1-3 h at 4-30° C. in a humidity condition of 20%-60%. In step B2, the dried and molded first microneedle 4 is pulled out along the side of the first microneedle 4 provided with the protrusions 42.

Since the inner molds 12 are arranged on the upper mold 1, on the substrate layer 5 of the molded microneedle patch, substrate through-holes 51 are formed corresponding to the positions where the inner molds 12 are arranged.

### Embodiment 13

On the basis of Embodiment 12, the microneedle mold provided in the present embodiment is used for molding a microneedle patch combined by a substrate layer 5 of a complete plate body and at least one first microneedle 4, and at least one protrusion 42 is provided on the first microneedle 4.

When the positioning insertion rods 13 of the present embodiment are inserted into the positioning slots and abut against the bottoms of the positioning slots 32, there is a gap between the upper mold 1 and the lower mold 3. When the inner molds 12 on the upper mold 1 are located in the first molding cavities 31 on the lower mold 3 and the first microneedles 4 are to be molded, the gap is used for air circulation, so as to accelerate the speed of the formulation liquid in the first molding cavities 31 being dried and molded into the first microneedles 4.

In some embodiments, the gap between the upper mold 1 and the lower mold 3 is 1 to 10 mm, and in some embodiments, the gap is 5 mm.

Further, the present embodiment also provides a preparation method for a microneedle patch. The microneedle patch is fabricated in two steps, that is, first microneedles 4 are molded first, and then the microneedle patch is prepared. The specific steps are as follows:
C1: mixing sodium hyaluronate and an efficacy material into a formulation liquid, and performing defoaming;
C2: spray-injecting the formulation liquid into first molding cavities 31 under a high pressure, and scraping the formulation liquid flat along the top of the lower mold 3, so that each of the first molding cavities 31 is filled with the formulation liquid;
C3: inserting the positioning insertion rods 13 into the positioning slots 32, and inserting the inner molds 12 of the upper mold 1 into the first molding cavities 31 on the lower mold 3;
C4: drying and molding first microneedles 4;
C5: separating the upper mold 1 from the lower mold 3; and

C6: attaching an adhesive substrate layer 5 to the top of the lower mold 3, so that the adhesive substrate layer 5 adheres to the end part of the end of the needle base 413 of each first microneedle 4, and separating the first microneedles 4 from the first molding cavities 31.

In some embodiments, in step C 4: drying for 1 h at 4° C. in a humidity condition of 20%.

### Embodiment 14

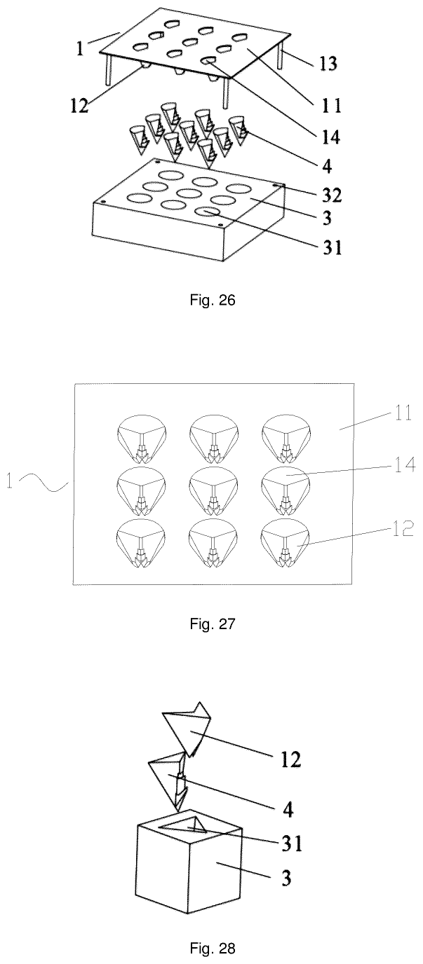

As shown in FIGS. 26-27, on the basis of Embodiment 13, the microneedle mold provided in the present embodiment is used for molding a microneedle patch combined by a substrate layer 5 of a complete plate body and at least one first microneedle 4, and at least one protrusion 42 is provided on the first microneedle 4.

In this embodiment, the upper mold bottom plate 11 is provided with at least one upper mold bottom plate through-hole 14, which is arranged at intervals, the upper mold bottom plate through-holes 14 are arranged adjacent to the inner molds 12, and the upper mold bottom plate through-holes 14 are arranged on the same side as the grooves 122 on the corresponding inner molds 12.

When the inner mold 12 is located in the first molding cavity 31, along the direction in which the lower mold 3 points to the upper mold 1, the space enclosed by the first molding cavity 31 and the inner mold 12 extends linearly and penetrates through the upper mold bottom plate 11 to form the upper mold bottom plate through-hole 14.

Further, in this embodiment, when the positioning insertion rods 13 are inserted into the positioning slots 32 and abut against the bottoms of the positioning slots, the upper mold 1 abuts against the lower mold 3, and the formulation liquid in the first molding cavities 31 is subjected to ventilation and drying through the upper mold plate through-holes 14.

Further, the present embodiment also provides a preparation method for a microneedle patch. The microneedle patch is fabricated in two steps, that is, first microneedles 4 are molded first, and then the microneedle patch is prepared.

The specific steps are as follows:
D1: mixing sodium hyaluronate and an efficacy material into a formulation liquid, and performing defoaming;
D2: inserting the positioning insertion rods 13 of the upper mold 1 into the positioning slots 32 of the lower mold 3 correspondingly, and inserting the inner mold 12 of the upper mold 1 into the first molding cavities 31 on the lower mold 3;
D3: the formulation liquid covering the surfaces of microneedle mold, performing centrifuging, injecting the formulation liquid into the first molding cavities 31 through the upper mold bottom plate through-holes 14, and scraping flat along the top of the upper mold 1 by using a scraper, so that each of the first molding cavities 31 is filled with the formulation liquid;
D4: drying and molding first microneedles 4;
D5: pulling the inner molds 12 out of the first molding cavities 31; and
D6: attaching an adhesive substrate layer 5 on the top of the lower mold 3, so that the adhesive substrate layer 5 adheres to the end part of the end of the needle base 413 of each first microneedle 4 away from the needle tip 411, and peeling off the substrate layer 5 from the side of the first microneedle 4 not provided with the protrusion 42 to the side of the first microneedle 4 provided with the protrusion 42, so as to separate the molded first microneedles 4 from the first molding cavities 31.

In some embodiments, in step D4, the drying conditions are: drying for 1.5 h at 25° C. in a humidity condition of 45%.

### Embodiment 15

As shown in FIG. 23, on the basis of Embodiment 12, the microneedle mold provided in the present embodiment is used for molding a microneedle patch combined by a substrate layer 5 provided with substrate through-holes 51 and at least one first microneedle 4, and at least one protrusion 42 is provided on the first microneedle 4.

The microneedle mold of this embodiment further includes a middle plate 2, wherein the middle plate 2 is located between the upper mold 1 and the lower mold 3, and the middle plate 2 is detachably connected to the upper mold 1 and the lower mold 3 respectively. A middle plate through-hole 21 is provided on the middle plate 2, the middle plate through-hole 21 is used for accommodating the inner molds 12, and is used for molding the substrate layer 5 provided with substrate through-holes 51.

Specifically, the middle plate 2 is provided with positioning holes 22, and the positioning holes 22 correspond to the positioning insertion rods 13 and the positioning slots 32.

In some embodiments, the middle plate 2 is made of other types of plastics such as PET (polyethylene terephthalate), and the thickness of the middle plate 2 is 0.1mm-2 mm.

Further, the structures of the positioning insertion rods 13 of the upper mold 1 and the positioning slots 32 of the lower mold 3 i this embodiment are the same as those in Embodiment 11.

Further, the present embodiment also provides a preparation method for a microneedle patch, wherein the microneedle patch is molded in one step, and the specific method is as follows:
E1: mixing sodium hyaluronate and an efficacy material into a formulation liquid, and performing defoaming;
E2: combining the middle plate 2 with the lower mold 3;
E3: spray-injecting the formulation liquid into the first molding cavities 31 under a high pressure, and scraping the formulation liquid flat along the middle plate 2 by using a scraper, so that the middle plate through-hole 21 is filled with the formulation liquid;
E4: inserting the positioning insertion rods 13 into the positioning slots 32, and inserting the inner mold 12 into the first molding cavities 31;
E5: drying and molding first microneedles 4 of which the top is provided with a substrate layer 5;
E6: pulling the inner molds 12 out of the first molding cavities 31, separating the middle plate 2 from the lower mold 3, and forming substrate through-holes 51 at positions on the substrate layer 5 corresponding to the inner molds 12; and
E7: peeling off the substrate layer 5 from the side of the first microneedle 4 not provided with the protrusion 42 to the side provided with the protrusion 42, to separate the first microneedles 4 from the first molding cavities 31, so as to fabricate the microneedle patch.

In some embodiments, in step E5, the drying conditions are: drying for 3 h at 4° C. in a humidity condition of 60%.

In the preparation process of the microneedle patch, the inner molds 12 are first pulled out from the first molding cavities 31, substrate through-holes 51 are formed on the substrate layer 5 of the microneedle patch, first microneedles 4 provided with protrusions 42 are still located in the first molding cavities 31, and a gap exists between the side of each microneedle body 41 provided with the protrusion 42 and the first molding cavity 31. When the first microneedles 4 are demolded, the substrate layer 5 is peeled off along the direction from the side of the first microneedle 4 not provided with the protrusion 42 to the side provided with the protrusion 42. The first microneedles 4 are integrally formed on the substrate layer 5, and thus the first microneedles 4 are detached from the first molding cavities 31 along with the substrate layer 5, to complete a demolding operation. The side of the substrate layer 5 away from the protrusion 42 is complete, has no hollows and stress concentration situation, the binding strength between the substrate layer 5 and the first microneedles 4 is high, and a gap exists between the lower mold 3 and the first microneedle 4 close to the side of the first microneedle 4 provided with the protrusion 42, and the resistance of separating the first microneedles 4 from the lower mold 3 is small; thus, when the first microneedles 4 are demolded, the substrate layer 5 can be smoothly separated from the silicone mold together with the first microneedles 4, to complete demolding, the process is convenient, the needles are not easy to be broken, and the yield is high.

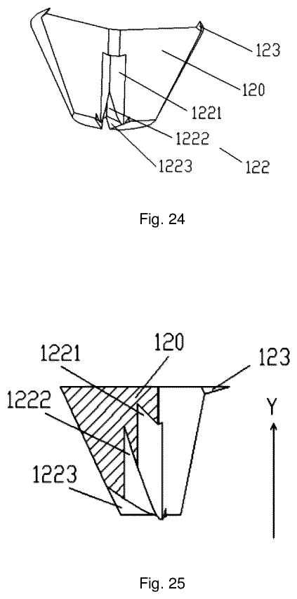

As shown in FIGS. 24 and 25, furthermore, guide port molds 123 are provided on each inner mold 12, so as to form guide ports 414 on the first microneedle 4 at a connection position between the first microneedle 4 and the substrate layer 5 after the first microneedle 4 is molded. The guide port molds 123 are protuberances provided on a large diameter end face of the inner mold body 120 and extend in the circumferential direction thereof. The length of a connecting line between two end points of open ends of the maximum diameter end face of the guide port molds 123 is greater than the length of a connecting line of two joint points respectively between the two guide ports 414 on the first microneedle 4 and the circumferential side wall of the needle base 413, so that tearing cracks generated when the substrate layer 5 is torn off can extend around two sides of the first microneedle 4 and then intersect.

### Embodiment 16

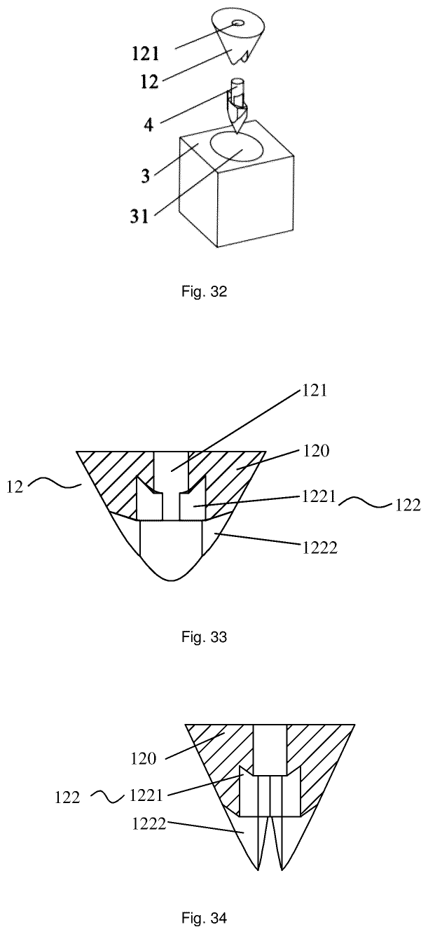

As shown in FIGS. 32-34, on the basis of Embodiment 2, the microneedle mold in the present embodiment is used for molding a microneedle patch which includes a substrate layer 5 of a complete plate body and first microneedles 4 each provided with at least one protrusion 42.

The microneedle mold in this embodiment sequentially includes an upper mold 1 and a lower mold 3 from top to bottom, wherein the upper mold 1 is detachably connected to the lower mold 3. The upper mold 1 includes an upper mold bottom plate 11, and at least one inner mold 12 is arranged at the bottom of the upper mold bottom plate 11. The top of the lower mold 3 is provided with at least one first molding cavity 31. Each molding cavity 31 corresponds to one inner mold 12, and the first molding cavity 31 accommodates the corresponding inner mold 12. The inner mold 12 is provided with a cavity 121 penetrating vertically, the cavity 121 is in communication with the first molding cavity 31, and the cavity 121 includes at least one groove 122. The space enclosed by the inner mold 12 and the first molding cavity 31 is used for molding the first microneedle 4, such that the first microneedle 4 is molded in the space.

After the first microneedles 4 are molded, the first the upper mold 1 is separated from the lower mold 3, and then an adhesive substrate layer 5 is adhered to the top of the lower mold 3, so that the adhesive substrate layer 5 adheres to the end part of the end of the needle base 413 of each first microneedle 4 away from the needle tip 411, and the first microneedles 4 are taken out from the first molding cavities 31, so as to prevent the protrusions 42 on the first microneedles 4 from being damaged during demolding.

In some embodiments, the cavity 121 includes two grooves 122, and the two grooves 122 are arranged opposite to each other, so as to form two opposite protrusions 42 on the first microneedle 4; or the cavity 121 includes four grooves 122, so as to mold four protrusions 42 arranged at intervals on the first microneedle 4 along the circumferential direction of the first microneedle.

In some embodiments, the horizontal projection of the inner mold 12 is circular or elliptical. When the horizontal projection of the inner mold 12 is circular, the first molding cavity 31 of the lower mold 3 is conical. When the horizontal projection of the inner mold 12 is elliptical, the first molding cavity 31 of the lower mold 3 is elliptic cone-shaped.

Further, in this embodiment, when the positioning insertion rods 13 are inserted into the positioning slots 32, the upper mold 1 abuts against the lower mold 3, and the formulation liquid in the first molding cavity 31 is subjected to ventilation and drying through the cavity 121.

The present embodiment also provides a preparation method for a microneedle patch, wherein the microneedle patch is fabricated in two steps, and the specific method is as follows:
F1: mixing sodium hyaluronate and an efficacy material into a formulation liquid;
F2: inserting the positioning insertion rods 13 into the positioning slots 32, and inserting the inner molds 12 into the first molding cavities 31;
F3: the formulation liquid covering the surfaces of microneedle mold, performing centrifuging, injecting the formulation liquid into the first molding cavities 31 through the cavities 121 of the inner molds 12, and scraping flat along the top of the upper mold 1 by using a scraper, so that each of the first molding cavities 31 is filled with the formulation liquid;
F4: drying and molding first microneedles 4;
F5: pulling the inner molds 12 out of the first molding cavities 31; and
F6: attaching a substrate layer 5 to the top of the lower mold 3, and peeling off the substrate layer 5 from the side of the first microneedle 4 not provided with the protrusion 42 to the side which provided with the protrusion 42, to separate the molded first microneedles 4 from the first molding cavities 31.

In some embodiments, in step F4, the drying conditions are: drying for 1.5 h at 25° C. in a humidity condition of 45%.

### Embodiment 17

On the basis of Embodiment 15, in this embodiment, the top of the lower mold 3 is further provided with at least one second molding cavity 33, and when the microneedle mold is closed, no inner mold 12 exists in the second molding cavity 33, so that a second microneedle 40 is molded in the second molding cavity 33, as shown in FIG. 36.

In this embodiment, the at least one first molding cavity 31 and the at least one second molding cavity 33 are arranged on the lower mold 3 at intervals. When the upper mold 1 is embedded with the lower mold 3, the inner mold 12 having the groove 122 is embedded in the first molding cavity 31, and no inner mold 12 is provided in the second molding cavity 33. The first molding cavity 31, the inner mold 12 embedded with the first molding cavity 31, and the second molding cavity 33 form a microneedle mold unit. The second molding cavity 33 is provided at one side of the first molding cavity 31, and when the upper mold 1 is embedded with the lower mold 3, the second molding cavity 33 is located at the side, provided with the groove 122, of the inner mold 12 embedded with the first molding cavity 31.

In some embodiments, a plurality of microneedle mold units are arranged in a manner of multiple rows and multiple columns, and when the upper mold 1 is embedded with the lower mold 3, the groove 122 of each inner mold 12 extends in the same direction so as to form a microneedle patch having a plurality of microneedle units, and corresponding each microneedle unit includes a first microneedle 4 and a second microneedle 40 which are arranged at an interval. The first microneedle 4 is provided with a protrusion 42, and the second microneedle 40 is a microneedle not provided with a protrusion 42. The second microneedle 40 is located on the side of the first microneedle 4 not provided with the protrusion 42. The arrangement directions of the microneedle units on the substrate layer 5 of the microneedle patch are the same, and the protrusions 42 of the first microneedles 40 are directed in the same direction; and in the microneedle mold unit, guide port molds 123 are provided on the inner mold 12, and the guide port molds 123 are protuberances extending in the circumferential direction and provided on a large diameter end face of the inner mold body 120. The length of a connecting line between two end points of open ends of the maximum diameter end face of the guide port molds 123 is greater than the length of a connecting line of two joint points respectively between the two guide ports 414 on the first microneedle 4 and the circumferential side wall of the needle base 413. The length of a connecting line of two joint points respectively between the two guide ports 414 on the first microneedle 4 and the circumferential side wall of the needle base 413 is greater than the maximum length of a connecting line of two ends, which are in the same direction as the connecting line of the two joint points, of an end face of the second molding cavity 33. The extension lines of the guide port molds 123 are tangent to the circumferential direction of the end face of the second molding cavity 33, so that the guide ports 414 are located on the circumferential side wall of the needle base 413 of the first microneedle 4; and the extension lines of the guide port molds 123 are tangent to the circumferential side wall of the needle base 413 of the second microneedle 40. The microneedle patch prepared by the mold unit provided above can enable the tearing cracks generated at the stress point structure to extend along the extension directions of the guide ports 414 and to be tangent to the circumferential side wall of the second microneedle 40 after the microneedles puncture into the skin and the substrate layer 5 is torn off; and under the continuous action of a tearing external force, the tearing cracks surround the microneedle unit and intersect at a gap between the microneedle unit and a microneedle unit adjacent thereto, to prevent the second microneedle 40 from being dragged out when the substrate layer 5 is torn off. Furthermore, a tearing crack region formed by each stress point structure is away from an adjacent microneedle unit, so as to avoid interference to the adjacent microneedle unit in the process of tearing off the substrate layer 5, and facilitate tearing of the substrate layer 5.

In some embodiments, the first molding cavity 31 has a radius R₁ and the second molding cavity 33 has a radius r₁, and then 0.17R₁≤r₁≤0.59R₁.

In some embodiments, the first molding cavity 31 is conical, and the maximum radius thereof is R₁, and along the connecting line direction between the center of circle of the first molding cavity 31 and the center of circle of the second molding cavity 33, the distance from the center of circle of the first molding cavity 31 of the microneedle mold unit to the center of circle of the first molding cavity 31 of an adjacent microneedle unit is L₁, then 3R₁≤L₁≤6R₁. In addition, the substrate layer 5 of the microneedle patch prepared by the microneedle mold unit has sufficient strength, after the microneedle patch is applied to the skin and the microneedles puncture into the skin, when the substrate layer 5 is torn off, a region of the substrate layer 5 not provided with the microneedles has sufficient strength such that the substrate layer 5 can be torn off once, which can effectively prevent the substrate layer 5 from being torn off for multiple times due to the breaking of the region not provided with the microneedles on the substrate layer 5.

Although embodiments of the present disclosure have been shown and described as above, it would be appreciated that the described embodiments are illustrative and cannot be construed to limit the present disclosure, and a person of ordinary skill in the art could make variations, amendments, replacements and modifications to the embodiments within the scope of the present disclosure.

What is claimed is:

1. A microneedle patch, comprising: a substrate layer and a first microneedle provided on the substrate layer, wherein a number of the first microneedle is at least one; each first microneedle comprises a microneedle body and at least one fixing portion provided on the microneedle body; the microneedle body comprises a needle base close to the substrate layer and a needle tip away from the substrate layer; and each fixing portion comprises at least one protrusion each protrusion is configured to extend in a radial direction of the first microneedle from the needle tip to the needle base, and the each protrusion has a tip end away from the microneedle body.

2. The microneedle patch according to claim 1, wherein a number of the at least one protrusion is greater than or equal to two, and along a direction in which the needle base of the each first microneedle extends towards the needle tip, a horizontal projection area and volume of a plurality of protrusions increase in sequence.

3. The microneedle patch according to claim 2, wherein an included angle between an extension direction of the each protrusion and an axis of the microneedle body is α, 15°≤α≤75°; or wherein when the number of the protrusions is three, along the direction in which the needle base of the each microneedle extends towards the needle tip, the protrusions sequentially comprise a first protrusion, a second protrusion and a third protrusion; and an included angle between an extension direction of the first protrusion and an axis of the microneedle body is 45°, an included angle between an extension direction of the second protrusion and the axis of the microneedle body is 30°, and an included angle between an extension direction of the third protrusion and the axis of the microneedle body is 60°; or
wherein when there are two protrusions, the two protrusions sequentially comprise a first protrusion and a second protrusion; a circumferential side wall of the first protrusion extends vertically, or is obliquely arranged in a direction close to the microneedle body; a circumferential side wall of the second protrusion extends vertically, or is obliquely arranged in a direction away from the microneedle body, and is configured to abut against a circumferential side wall of a mold molding cavity; and
when there are three protrusions, the three protrusions sequentially comprise a first protrusion, a second protrusion and a third protrusion; a circumferential side wall of the first protrusion extends vertically; a circumferential side wall of the second protrusion extends vertically, or is obliquely arranged in a direction close to the microneedle body; and a circumferential side wall of the third protrusion extends vertically, or is obliquely arranged in a direction away from the microneedle body, and is configured to abut against a circumferential side wall of the mold molding cavity.

4. (canceled)

5. (canceled)

6. The microneedle patch according to claim 1, wherein a horizontal projection of the needle base in the each first microneedle is fan-shaped and the microneedle body in the each first microneedle is a cone with a horizontal projection in a fan shape, the at least one protrusion is located on an edge of the microneedle body connected to the tip end of a fan-shaped flat surface, and protrusions on the microneedle body face the same direction;
or the microneedle body is a cone with a horizontal projection in an elliptic fan shape, the at least one protrusion is provided on an edge of the microneedle body connected to the tip end of an elliptic fan-shaped bottom face and is located on a short-axis side of a horizontal projection of the microneedle body;
or the microneedle body is a pyramid, and the at least one protrusion is located on an edge of the microneedle body; or
wherein the microneedle patch further comprises a second microneedle; at least one first microneedle and at least one second microneedle arranged at an interval form a microneedle unit; the microneedle patch comprises at least one microneedle unit, the second microneedle is a microneedle not provided with the fixing portion, and the second microneedle is located on a side of the each first microneedle not provided with the fixing portion.

7. (canceled)

8. The microneedle patch according to claim 1, wherein a stress point structure for tearing the substrate layer and separating the each first microneedle from the substrate layer is provided at a connection position between the each first microneedle and the substrate layer; the stress point structure comprises a substrate through-hole provided on the substrate layer and on the same side as the fixing portion on the each first microneedle.

9. The microneedle patch according to claim 8, wherein the stress point structure further comprises guide ports the guide ports are located at junctions of the needle base of the first microneedle and the substrate through-hole, and the guide ports are located on a circumferential side wall of the needle base or on the substrate layer.

10. The microneedle patch according to claim 9, wherein a minimum length of a connecting line of the guide ports on two sides of the first microneedle is greater than a maximum length of a second microneedle in a direction parallel to the connecting line of the guide ports.

11. The microneedle patch according to claim 8, wherein after the first microneedle punctures into the skin and when the substrate layer is torn off, the first microneedle and the substrate through-hole adjacent to the first microneedle form a group of stress unit, and at least one group of stress unit is arranged at interval on the substrate layer; the substrate layer and the first microneedle are separated by the stress point structure to form cracks, the cracks intersect on the substrate layer between two adjacent groups of stress units;
or after the microneedle unit punctures into the skin and when the substrate layer is torn off, the microneedle unit and the substrate through-hole adjacent thereto form a group of stress unit, and at least one group of stress unit is arranged at interval on the substrate layer; after the microneedle punctures into the skin and when the substrate layer is torn off, the cracks formed by the substrate layer being separated from the microneedle unit by the stress point structure intersect at a gap between two adjacent groups of microneedle units.

12. The microneedle patch according to claim 9, wherein the stress point structure further comprises guide ports; cracks formed by separating the substrate layer from the microneedle unit extend along the guide ports from the first microneedle to a second microneedle, and extends along a circumferential side wall of the needle base of the second microneedle, so that the second microneedle is separated from the substrate layer.

13. The microneedle patch according to claim 12, wherein extension lines of the guide ports intersect with or are tangent to a circumferential side wall of the needle base (413) of the second microneedle.

14. A microneedle mold, applied to manufacturing the microneedle patch according to claim 1, wherein the microneedle mold sequentially comprises an upper mold and a lower mold from top to bottom, the upper mold is detachably connected to the lower mold, the upper mold comprises an upper mold bottom plate, and at least one inner mold is provided at a bottom of the upper mold bottom plate;
at least one first molding cavity is provided at a top of the lower mold, each first molding cavity is provided corresponding to the at least one inner mold, and the each first molding cavity accommodates a corresponding inner mold;
each inner mold comprises an inner mold body and at least one groove provided on the inner mold body, and each groove is used for forming the each protrusion on a circumferential surface of the each first microneedle; and
a space enclosed by the each first molding cavity and the each inner mold is used for molding the first microneedle.

15. The microneedle mold according to claim 14, wherein the each inner mold is provided with guide port molds to form guide ports on the microneedle which provided with protrusions.

16. The microneedle mold according to claim 15, wherein the guide port molds are protuberances provided on the each inner mold body close to a maximum diameter end face and extending in a circumferential direction, so as to form the guide ports on the first microneedle provided with the protrusions; or wherein a length of a connecting line between two ends of open ends of a maximum diameter end face of the guide port molds provided on the inner mold body is greater than a length of a connecting line of two joint points between the two quide ports on the microneedle and an outer circumferential side wall of the needle base on the microneedle provided with the protrusions.

17. (canceled)

18. The microneedle mold according to claim 14, wherein a direction from an end with a smallest diameter to an end with a largest diameter of the inner mold body is a Y direction, and the each groove extends along the Y direction.

19. The microneedle mold according to claim 18, wherein there are at least two grooves, and along a direction opposite to the Y direction, volumes of the grooves increase sequentially, so that the volumes of corresponding protrusions in the molded microneedle increase sequentially.

20. The microneedle mold according to claim 14, wherein the microneedle mold further comprises a middle plate, wherein the middle plate is located between the upper mold and the lower mold, the middle plate is provided with a middle plate through-hole extending vertically, the middle plate through-hole accommodates the at least one inner mold, the middle plate through-hole is used to form the substrate layer, and the substrate layer and the microneedle form a microneedle patch.

21. The microneedle mold according to claim 14, wherein at least one second molding cavity is further provided at the top of the lower mold, and when the microneedle mold is closed, no inner mold is provided in each second molding cavity; and
a space in the each second molding cavity is used for molding a second microneedle not provided with the protrusion.

22. The microneedle mold according to claim 21, wherein the first molding cavity, the at least one inner mold embedded with the first molding cavity, and the second molding cavity form a microneedle mold unit, the microneedle mold unit is used for molding a microneedle unit, and when the microneedle mold is closed, the second molding cavity is located at a side, provided with the groove, of the each inner mold embedded with the first molding cavity.

23. The microneedle mold according to claim 14, wherein the upper mold bottom plate is provided with at least one upper mold bottom plate through-hole, each upper mold bottom plate through-hole is provided adjacent to a corresponding inner mold, and is provided at a same side as the groove on the inner mold;
when the inner mold is located in the first molding cavity, along a direction in which the lower mold extends towards the upper mold, a space enclosed by the first molding cavity and the inner mold extends linearly and penetrates through the upper mold bottom plate to form the each upper mold bottom plate through-hole.

24. The microneedle mold according to claim 21, wherein the first molding cavity has a radius R₁ and the second molding cavity has a radius r₁, and then 0.17R₁≤r₁≤0.59R₁.

25. The microneedle mold according to claim 24, wherein along a connecting line direction between a center of circle of the first molding cavity and a center of circle of the second molding cavity, a distance from the center of circle of the first molding cavity of a microneedle mold unit to the center of circle of the first molding cavity of an adjacent microneedle unit is L₁, then 3R₁≤L₁≤6R₁.

26. A preparation method for a microneedle patch, prepared by using the microneedle mold according to claim 14, wherein the preparation method comprises following steps:
injecting a formulation liquid into a microneedle molding cavity formed by the upper mold and the lower mold; and
drying for 1-3 h at 4-30° C. under a humidity condition of 20%-60%, so as to dry and mold the microneedle;
wherein the microneedle molding cavity at least comprises the first molding cavity, the first molding cavity accommodates the inner mold on the upper mold, and the inner mold is used for molding protrusions on the first microneedle.

27. The preparation method for the microneedle patch according to claim 26, wherein injecting a formulation liquid into the microneedle molding cavity formed by the upper mold and the lower mold comprises following steps:
S1: injecting the formulation liquid into the first molding cavity; and
S2: combining the upper mold and the lower mold, so that the inner mold is inserted into the first molding cavity; or,
S1: combining the upper mold and the lower mold, so that the inner mold is inserted into the first molding cavity; and
S2: injecting the formulation liquid into a space enclosed by the first molding cavity and the inner mold.

28. The preparation method for the microneedle patch according to claim 27, wherein step S2 further comprises:
providing a middle plate between the upper mold and the lower mold, a middle plate through-hole on the middle plate accommodating the inner mold, injecting the formulation liquid into the middle plate through-hole, and the formulation liquid in the middle plate through-hole drying and molding a substrate layer;
the formulation liquid in a space enclosed by the first molding cavity and the inner mold drying and molding the microneedle provided with the protrusions;
pulling the dried microneedle provided with the protrusions out of the first molding cavity; and
when the microneedle provided with the protrusions is pulled out of the first molding cavity, peeling off the substrate layer from a side of the microneedle not provided with the protrusions to a side of the microneedle provided with the protrusions, to separate the microneedle provided with the protrusions from the first molding cavity, so as to mold a microneedle patch.

29. The preparation method for the microneedle patch according to claim 27, wherein step S2 further comprises:
drying and molding the microneedle provided with the protrusions;
pulling the dried microneedle provided with the protrusions out of the first molding cavity; and
when the microneedle provided with the protrusions is pulled out of the first molding cavity, attaching an adhesive substrate layer to a top of the lower mold, so that the adhesive substrate layer adheres to an end part of the microneedle away from the needle tip and peeling off the adhesive substrate layer from a side of the microneedle not provided with the protrusions to a side of the microneedle provided with the protrusions, to separate the microneedle provided with the protrusions from the first molding cavity, so as to mold a microneedle patch.
